SEQUENCE LISTING (7 sequences)

SEQ ID NO: 1 - protein, 645 aa, Homo sapiens
MSERKEGRGKGKGKKKERGSGKKPESAAGSQSPALPPRLKEMKSQESAAGSKLVLRCETSSEYSSLRFKWFKNGNELNRKNKPQNIKIQKKPGKSELRINKASLADSGEYMCKVISKLGNDSASANITIVESNEIITGMPASTEGAYVSSESPIRISVSTEGANTSSSTSTSTTGTSHLVKCAEKEKTFCVNGGECFMVKDLSNPSRYLCKCPNEFTGDRCQNYVMASFYKHLGIEFMEAEELYQKRVLTITGICIALLVVGIMCVVAYCKTKKQRKKLHDRLRQSLRSERNNMMNIANGPHHPNPPPENVQLVNQYVSKNVISSEHIVEREAETSFSTSHYTSTAHHSTTVTQTPSHSWSNGHTESILSESHSVIVMSSVENSRHSSPTGGPRGRLNGTGGPRECNSFLRHARETPDSYRDSPHSERYVSAMTTPARMSPVDFHTPSSPKSPPSEMSPPVSSMTVSMPSMAVSPFMEEERPLLLVTPPRLREKKFDHHPQQFSSFHHNPAHDSNSLPASPLRIVEDEEYETTQEYEPAQEPVKKLANSRRAKRTKPNGHIANRLEVDSNTSSQSSNSESETEDERVGEDTPFLGIQNPLAASLEATPAFRLADSRTNPAGRFSTQEEIQARLSSVIANQDPIAV

SEQ ID NO: 2 - protein, 31 aa, Artificial
  EGF-like domains of neuregulin
VNGGECFMVKDLSNPSRYLCKCPNEFTGDRC

SEQ ID NO: 3 - protein, 245 aa, Artificial
  Amino acids 1-245 of neuregulin
MSERKEGRGKGKGKKKERGSGKKPESAAGSQSPALPPRLKEMKSQESAAGSKLVLRCETSSEYSSLRFKWFKNGNELNRKNKPQNIKIQKKPGKSELRINKASLADSGEYMCKVISKLGNDSASANITIVESNEIITGMPASTEGAYVSSESPIRISVSTEGANTSSSTSTSTTGTSHLVKCAEKEKTFCVNGGECFMVKDLSNPSRYLCKCPNEFTGDRCQNYVMASFYKHLGIEFMEAEELYQ

SEQ ID NO: 4 - protein, 245 aa, Artificial
  Amino acids 2-246 of neuregulin
SERKEGRGKGKGKKKERGSGKKPESAAGSQSPALPPRLKEMKSQESAAGSKLVLRCETSSEYSSLRFKWFKNGNELNRKNKPQNIKIQKKPGKSELRINKASLADSGEYMCKVISKLGNDSASANITIVESNEIITGMPASTEGAYVSSESPIRISVSTEGANTSSSTSTSTTGTSHLVKCAEKEKTFCVNGGECFMVKDLSNPSRYLCKCPNEFTGDRCQNYVMASFYKHLGIEFMEAEELYQK

SEQ ID NO: 5 - protein, 71 aa, Artificial
  Amino acids 176-246 of neuregulin
TSHLVKCAEKEKTFCVNGGECFMVKDLSNPSRYLCKCPNEFTGDRCQNYVMASFYKHLGIEFMEAEELYQK

SEQ ID NO: 6 - protein, 61 aa, Artificial
  Amino acids 177-237 of neuregulin
SHLVKCAEKEKTFCVNGGECFMVKDLSNPSRYLCKCPNEFTGDRCQNYVMASFYKAEELYQ

SEQ ID NO: 7 - protein, 671 aa, Homo sapiens
MGHHHHHHHHHHSSGHIEGRHMRNNHYDKILAHSRIRGRDQGPNVCALQQILGTKKKYFSTCKNWYKKSICGQKTTVLYECCPGYMRMEGMKGCPAVLPIDHVYGTLGIVGATTTQRYSDASKLREEIEGKGSFTYFAPSNEAWDNLDSDIRRGLESNVNVELLNALHSHMINKRMLTKDLKNGMIIPSMYNNLGLFINHYPNGVVTVNCARIIHGNQIATNGVVHVIDRVLTQIGTSIQDFIEAEDDLSSFRAAAITSDILEALGRDGHFTLFAPTNEAFEKLPRGVLERFMGDKVASEALMKYHILNTLQCSESIMGGAVFETLEGNTIEIGCDGDSITVNGIKMVNKKDIVTNNGVIHLIDQVLIPDSAKQVIELAGKQQTTFTDLVAQLGLASALRPDGEYTLLAPVNNAFSDDTLSMVQRLLKLILQNHILKVKVGLNELYNGQILETIGGKQLRVFVYRTAVCIENSCMEKGSKQGRNGAIHIFREIIKPAEKSLHEKLKQDKRFSTFLSLLEAADLKELLTQPGDWTLFVPTNDAFKGMTSEEKEILIRDKNALQNIILYHLTPGVFIGKGFEPGVTNILKTTQGSKIFLKEVNDTLLVNELKSKESDIMTTNGVIHVVDKLLYPADTPVGNDQLLEILNKLIKYIQIKFVRGSTFKEIPVTVY

(19) United States
(12) Patent Application Publication
(10) Pub. No.: US 2012/0121557 A1
(43) Pub. Date: May 17, 2012

(54) NEUREGULIN INDUCED PROLIFERATION OF CARDIOMYOCYTES

(75) Inventor: Bernhard Kuhn, Brookline, MA (US)

(73) Assignee: CHILDREN'S MEDICAL CENTER CORPORATION, Boston, MA (US)

(21) Appl. No.: 13/386,187

(22) PCT Filed: Jul. 20, 2010
(86) PCT No.: PCT/US2010/042565

§ 371 Date: Jan. 20, 2012

Related U.S. Application Data
(60) Provisional application No. 61/227,677, filed on Jul. 22, 2009.

(51) Int. Cl. A61K 35/34; A61K 38/22; A61P 9/00; C12N 5/077

(52) U.S. Cl. 424/93.7; 435/376; 514/9.6; 435/375

(57) ABSTRACT

The present invention provides methods for inducing division of postmitotic mammalian differentiated cardiomyocytes. The invention can be used to repair heart tissue damaged by, for example, myocardial ischemia, hypoxia, stroke, myocardial infarction or chronic ischemic heart disease in vivo. In addition, the methods of the invention can be used to induce heart muscle cells to divide in vitro, in vivo and/or ex vivo, which can then be used in heart tissue repair.

### FIELD OF THE INVENTION

The human heart is incapable of adequate regeneration or repair after injury. Thus the invention discloses methods for inducing division of post-mitotic cells for repairing heart tissue.

### BACKGROUND OF THE INVENTION

Cardiovascular diseases are a leading cause of death, resulting in almost 40% of deaths annually in the United States. Inadequate human myocardial regeneration poses a significant public health problem. It is estimated that 13 million Americans have coronary artery disease, and more than half a million experience a myocardial infarction every year. Human cardiac tissue responds to injury, e.g. myocardial infarction, with scar formation. Because the human heart is incapable of adequate muscle regeneration, survivors of a myocardial infarction typically develop heart failure, arrhythmias, thrombosis, and other complications.

Adult human hearts do not regenerate after injury; instead, the defect is replaced by fibrotic tissue. Most evidence to date indicates that cardiomyocyte proliferation, the cellular basis of regeneration, is not a significant component of the mammalian response to acute injury. In contrast to adult cardiomyocytes, fetal cardiomyocytes do proliferate during development.

Heart disease results in the loss of cardiomyocytes. It has been a significant challenge to develop effective treatments for cardiac repair because adult mammalian cardiomyocytes are highly differentiated cells and presumed to be essentially unable to proliferate. Mammalian cardiomyocytes withdraw from the cell cycle soon after birth and have lowered levels of cyclin A (Yoshizumi, M., et. al. (1995). J Clin Invest 95, 2275-2280). The fact that primary cardiac tumors occur rarely supports the notion that adult cardiomyocytes are highly restricted in their ability to divide. Because of its lack of proliferative potential, the primary response of the mammalian heart to injury is scar formation, which prevents cardiac repair. Thus the loss of cardiomyocytes after damage caused by events such as myocardial infarction generally results in compensatory responses that are inadequate to restore function. Unreplaced loss of cardiomyocytes leads to heart failure, a significant health problem worldwide.

Current therapies are also limited in their effectiveness. In order to sufficiently repair cardiac injury, it would be highly desirable to provide the heart with new cardiomyocytes. Proliferation of endogenous cardiomyocytes could enhance the regenerative capacity of mammalian hearts.

Accordingly, there is a need in the art for methods of increasing and/or promoting proliferation of adult mammalian cardiomyocytes.

### SUMMARY OF THE INVENTION

The present invention provides methods and compositions for increasing proliferation, increasing cell cycle activity, and/or inducing division of post-mitotic mammalian differentiated cardiomyocytes. The invention can be used to slow, reduce, prevent or treat the onset of cardiac damage caused by, for example, myocardial ischemia, hypoxia, stroke, or myocardial infarction in vivo. The invention can also be used in a subject with chronic ischemic heart disease. In addition, the methods of the invention can be used in pharmaceutical compositions to enhance proliferation of differentiated cardiomyocytes in vitro and/or in vivo, or can be used ex vivo in tissue grafting.

The invention is based, in part, on the discovery that neuregulin, a component of the extracellular matrix, and fragments thereof promote differentiated cardiomyocytes to proliferate and facilitate myocardial regeneration. The adult mammalian heart responds to injury with scar formation, not with proliferation, the cellular basis for regeneration. The insufficient regeneration of mammalian hearts is explained by the contractile apparatus impinging on cardiomyocyte division. The invention demonstrates that extracellular neuregulin can induce cell cycle re-entry of differentiated mammalian cardiomyocytes.

Neuregulin stimulates mononuclear cardiomyocytes, present in the adult mammalian heart, to undergo the full mitotic cell cycle division. Without being limited to any particular mechanism of action, neuregulin is understood to activate ErbB4 located in the cardiomyocyte cell membrane. Neuregulin-induced cardiomyocyte proliferation results from activation of ErbB4 tyrosine kinase signaling pathways. After myocardial infarction, recombinant neuregulin induces cardiomyocyte cell cycle re-entry, improves cardiac remodeling and function, reduces fibrosis and infarct size, and increases angiogenesis. These results demonstrate that neuregulin and the pathways it regulates are new targets for innovative strategies to treat injured heart tissue.

In another aspect, the invention discloses methods of inducing division of a post mitotic cell comprising administering neuregulin to the cell in an amount and regime effective to stimulate mitotic division of the cell. The post-mitotic cells can be heart muscle cells/cardiomyocytes, and preferably mammalian heart muscle cells. In some embodiments, inducing division comprises at least one of inducing the heart muscle cell to reenter cell cycle, increasing DNA synthesis and inducing cytokinesis in the heart muscle cell.

The neuregulin composition can also be formulated into a pharmaceutical composition with a pharmaceutically acceptable carrier, diluent or medium for treating damaged heart tissue. The neuregulin composition of the invention can further comprise at least a fragment of the neuregulin composition of SEQ ID NO:1 or a sequence that is at least 80%, 85%, 90%, 91%, 92%, 93%, 94%, 95%, 96%, 97%, 98%, 99% identical to that of the SEQ ID NO:1 fragment. In another embodiment, the neuregulin composition comprises a polypeptide comprising the neuregulin fragment of SEQ ID NO:2 or a functional variant or a sequence that is at least 80%, 85%, 90%, 91%, 92%, 93%, 94%, 95%, 96%, 97%, 98%, 99% identical to that of SEQ ID NO:2. In other embodiments, the neuregulin composition can comprise a polypeptide comprising the neuregulin fragment of SEQ ID NO:3, SEQ ID NO:4, SEQ ID NO:5, or SEQ ID NO:6 or a functional variants thereof or a sequence that is at least 80%, 85%, 90%, 91%, 92%, 93%, 94%, 95%, 96%, 97%, 98%, 99% identical to that of SEQ ID NO:3, SEQ ID NO:4, SEQ ID NO:5, or SEQ ID NO:6. In yet another embodiment, the neuregulin composition can comprise at least an epidermal growth factor-like (EGF-like) domain of neuregulin and the neuregulin can activate ErbB4 or a fragment that is at least 80%, 85%, 90%, 91%, 92%, 93%, 94%, 95%, 96%, 97%, 98%, 99% identical to an epidermal growth factor-like domain of neuregulin and the fragment can activate ErbB4. The neuregulin composition can also induce or facilitate heterodimerization of ErbB4 and ErbB2 receptors or homodimerization of ErbB4 receptors.

In another embodiment, the neuregulin composition can be administered in an amount and regime effective to stimulate mitotic division. The administration regime can be a duration sufficient to induce cell cycle re-entry of the heart muscle cells. Data has shown that administration for at least 12 weeks can stimulate division by inducing the heart muscle cells to re-enter the cell cycle, increase DNA synthesis and induce cytokinesis.

In another aspect of the invention, cardiomyocytes can be induced to proliferate by selecting differentiated cells from a tissue that includes the differentiated cells. The cells can further be resuspended in a growth medium containing an effective amount of a neuregulin composition, e.g. comprising an epidermal growth factor-like domain of neuregulin. The differentiated cells can be cultured in the neuregulin growth medium for a time and under appropriate conditions to induce proliferation of at least a portion of the cultured cells, wherein at least a portion of the differentiated cells in culture undergo at least one round of cardiomyocyte division.

The method of inducing the cells in vitro can further comprise transplanting the proliferating cardiomyocytes. The cells can be seeded on a biodegradable scaffold. The cells can also be directly transplanted into a target area of a subject, wherein the target area can be a damaged heart tissue. The proliferating cardiomyocytes can also be incorporated into a heart tissue transplant, wherein the transplant can be transplanted into a target area of the subject, such as a damaged heart tissue.

In another aspect, the invention provides a method of repairing heart tissue, comprising identifying a subject in need of heart tissue repair, administering to the subject an effective amount of a neuregulin composition, in an amount and regime effective to stimulate division of post-mitotic cardiomyocytes, and inducing proliferation of the cardiomyocytes to thereby repair heart tissue. The neuregulin can be formulated and delivered by a route selected from the group consisting of a parenterally, an orally, an intraperitoneally, an intravenously, a catheter infusion, an inhalation and a transdermal application. The invention can also comprise delivering neuregulin to a target area of the heart tissue. The neuregulin can be delivered locally to the target area or systemically through methods such as catheter infusion or intravenously. Local and/or targeted delivery can also be administered using a slow controlled release delivery system, such as, for example, a biodegradable matrix. The invention can also be used with a long-term, short-term and/or controlled release delivery systems.

In some embodiments, the subject in need of heart tissue repair has undergone myocardial ischemia, hypoxia, stroke, or myocardial infarction. The method of repairing heart tissue can also comprise replacing damaged heart tissue with proliferating cardiomyocytes, improving myocardial function in the subject and reducing myocardial hypertropy to repair the heart tissue.

In another aspect, the invention provides a method for treating a condition or disease state by stimulating proliferation of post-mitotic cells comprising administering a compound comprising a neuregulin composition or a pharmaceutically acceptable derivative thereof, whereby the compound treats the condition or disease state by stimulating proliferation of the post-mitotic cells.

Further understanding of various aspects of the invention can be obtained by reference to the following detailed description in conjunction with the associated drawings, which are described briefly below.

### BRIEF DESCRIPTION OF THE DRAWINGS

FIG. 1. NRG1 induces cell cycle reentry and division of differentiated cardiomyocytes in vitro. Primary adult rat ventricular cardiomyocytes were stimulated, labelled with BrdU for the last 3 days, and DNA synthesis was determined by immunofluorescence microscopy after 9 days. (A) NRG1 (100 ng/mL), fibroblast growth factor 1 (FGF1, 100 ng/mL), and periostin (500 ng/mL) induce cardiomyocyte DNA synthesis, while epidermal growth factor (EGF), heparin-binding epidermal growth factor (HB-EGF), or platelet-derived growth factor BB (PDGF, 100 ng/mL each) do not. Results were normalized to NRG1=100%. (B) NRG1-stimulated cardiomyocyte DNA synthesis was concentration-dependent. (C) NRG1-stimulated (125 pM) cardiomyocyte DNA synthesis inhibited with increasing concentrations of an antibody against ErbB2. (D) Functional inhibition of PI3K with PTEN attenuates NRG1-stimulated DNA synthesis in cardiomyocytes. (E,F) Fate mapping of individual cardiomyocytes showing a portion of cardiomyocytes performing DNA synthesis over a period of 3 days (E) and that were in cytokinesis on day 9 (F). (G) Cardiomyocyte DNA synthesis precedes cytokinesis;

FIG. 2. The epidermal growth factor-like domain of NRG1 is sufficient to induce cardiomyocyte cell cycle reentry. Primary adult rat ventricular cardiomyocytes were stimulated, labeled with BrdU for the last 3 days, and DNA synthesis was determined by immunofluorescence microscopy after 9 days. No stim., no stimulation. NRG1 EGF domain, human NRG1 epidermal growth factor-like domain (amino acids 176-246); NRG1 EC domain, NRG1 extracellular domain (amino acids 1-246);

FIG. 3. Differential proliferative potential of mono- and binucleated cardiomyocytes are depicted. Pie chart on left shows the relative number of cardiomyocytes analyzed and the respective number of mono- and binucleated cardiomyocytes observed (100%). Pie chart of left, drawn to scale of the number of observed cardiomyocytes, shows the relative number of cardiomyocytes that completed cytokinesis (0.6±0.3%).

FIG. 4. ErbB4 controls postnatal cardiomyocyte proliferation in vivo. (A-C) Experiments were performed in α-MHC-MerCreMer^{+/+}; ErbB4^{F/F} (test group) and in α-MHC-MerCreMer^{+/+}; ErbB4^{Wt/F} (control group) mice. (A) Inactivation of ErbB4 does not affect portion of mono- and multinucleated cardiomyocytes. (B,C) Inactivation of ErbB4 abolishes postnatal cardiomyocyte cell cycle activity (B) and disrupts cardiomyocyte proliferation (C). (D) Transgenic expression of ErbB4 under control of the α-MHC promoter does not change the portion of mono- and multinucleated cardiomyocytes. (E) Overexpressing ErbB4 in differentiated cardiomyocytes increases cell cycle activity of mononucleated cardiomyocytes. (F) Overexpressing ErbB4 has no effect on cardiomyocyte apoptosis. Positive control is 1 week after myocardial infarction. (G,H) ErbB4-induced cardiomyocyte cell cycle activity results in more (G) and smaller (H) cardiomyocytes. Scale bars 25 μm. Significance tested by ANOVA (A,B,E) and t-test (C,F-H). Results are means±s.e.m. from more than 8 different hearts per experiment;

FIG. 5. NRG1 induces cycling of differentiated cardiomyocytes in vivo in an ErbB4-dependent mechanism. (A) Experimental design. Vertical arrowheads indicate daily NRG1 injections. (B) ErbB4 controls NRG1-induced cardiomyocyte cell cycling. (C) Proportions of mono-, bi-, and multinucleated cardiomyocytes are not affected by modulating NRG1/ErbB4 signaling. (D, E) NRG1 induces cardiomyocyte karyokinesis (D) and cytokinesis (E, Aurora B-kinase-positive midbody shown in a series of XZ reconstructions). Results are means±s.e.m. from at least 5 animals per experiment;

FIG. 6. NRG1 induces proliferation of differentiated cardiomyocytes in vivo. Experimental design for genetic fate map using α-MHC-MerCreMer^{+/+}; Rosa26R mice^{+/-}. Injections of tamoxifen (Tam) to induce permanent genetic labeling and NRG1-induced DNA synthesis and karyokinesis originate from differentiated cardiomyocytes. (A) Quantification of cardiomyocyte clusters after 9 NRG1-injections. Repeated transition through the S-phase of the cell cycle was detected by successively labelling with the thymidine analogs chlorodeoxyuridine (CldU) and iododeoxyridine (IdU). (B) Diagram of portion of cardiomyocytes by frequency of cell cycle transitions and number of nuclei;

FIG. 7. Undifferentiated progenitor cells do not contribute to NRG1-induced cardiomyocyte cell cycle activity. Differentiated cardiomyocytes were genetically labelled by activation of β-galactosidase transcription, visualized by X-gal staining. Cardiomyocyte proliferation was induced by injecting NRG1 into adult mice (2 mo. of age). (A) Stepwise incremental genetic labelling demonstrates lack of correlation between genetic labelling frequency and NRG1-induced cardiomyocyte generation, thus indicating that genetically labelled and unlabelled cardiomyocytes originate from differentiated cardiomyocytes. (B) Regression of NRG1-induced cardiomyocyte generation and recombination frequency shows lack of correlation between genetic labelling frequency and cell cycle activity in the genetically unlabelled fraction, indicating that NRG1-induced cardiomyocyte proliferation originates from differentiated cardiomyocytes. Color codes indicated at the top. Scale bars 50 μm. Statistical significance was determined by ANOVA (A) and by linear regression (B). Results are means±s.e.m. from more than 11 animals per experiment;

FIG. 8. NRG1 treatment improves myocardial function and induces scar regression. Myocardial infarction was induced at 2 months of age. NRG1 or vehicle injections were begun one week later and continued for 1 or for 12 weeks. All mice were treated with BrdU in the drinking water during the final week of injections as indicated by the green arrow. Animals in the 12-week treatment arm were euthanized 2 weeks later to determine whether NRG1-effects were permanent. (A) NRG1 treatment improves ventricular remodelling and myocardial function as shown by echocardiographic measurements of left ventricular internal dimensions (LVID), interventricular septum (IVS), left ventricular posterior wall (LVPW) and ejection fraction (EF). Quantification of infarct scar of control and NRG1-treated hearts (B). (C) Heart weight is not affected by NRG1 treatment. (D) Cardiomyocyte cross sectional area is lower in NRG1-treated hearts. Statistical significance determined by ANOVA (A,B,C) and t-test (D). Results are means±s.e.m. from 10-32 animals;

FIG. 9. NRG1 promotes cardiomyocyte proliferation after myocardial infarction. (A) Quantification of cardiomyocyte DNA synthesis after 1 week of continuous labelling with BrdU. Representative BrdU-positive cardiomyocyte in scar region. (B) NRG1 treatment does not affect cardiomyocyte apoptosis. (C) NRG1-treatment does not affect percentage of mono- and multinucleated cardiomyocytes. (D) Quantification of cardiomyocyte mitoses by visualization of metaphase chromosomes. H3P-positive cardiomyocyte in scar region. (E) Quantification of cardiomyocyte cytokineses by visualization of the contractile ring. (F) Quantification of left ventricular cardiomyocyte nuclei shows significant cardiomyocyte replacement after 12 weeks of NRG1 treatment. (G) Quantification of X-gal positive and negative differentiated mononucleated cardiomyocytes that had undergone DNA synthesis, which have identical morphology, suggesting similar cellular origins. Results are means±s.e.m. from 11-24 animals;

FIG. 10. Molecular model of cardiomyocyte proliferation;

FIG. 11. Cellular model cardiomyocyte proliferation.

### DETAILED DESCRIPTION OF THE INVENTION

Humans do not regenerate their hearts after injury; instead, the defect is replaced by fibrotic tissue. The inadequate regenerative response of injured human hearts contributes significantly to morbidity and mortality from cardiovascular diseases, such as myocardial infarction (MI). By contrast, lower vertebrates, such as newt and zebrafish, regenerate their hearts by cardiomyocyte proliferation. The invention discloses that cardiomyocyte proliferation, the cellular basis of regeneration, can be stimulated by neuregulin and biologically active fragments thereof.

Cardiomyocytes proliferate during prenatal development (Pasumarthi, K. B., and Field, L. J. (2002). Cardiomyocyte cell cycle regulation. Circ Res 90, 1044-1054). Soon after birth, however, cardiomyocytes become binucleated and withdraw from the cell cycle, giving rise to the notion that adult cardiomyocytes are incapable of proliferating, i.e. they are terminally differentiated. Specifically, cardiomyocytes in the adult mammalian heart are thought to be incapable of performing cytokinesis, the ultimate step of the mitotic cell cycle (Ahuja, P., Sdek, P., and MacLellan, W. R. (2007). Cardiac myocyte cell cycle control in development, disease, and regeneration. Physiol Rev 87, 521-544).

Differentiated cardiomyocytes or heart muscle cells can be induced to proliferate by activating specific signaling pathways, leading to enhanced myocardial regeneration and improved heart function. These findings offer a new strategy to promote the repair process after myocardial infarction. Since specific organ functions rely on differentiated cells, replacing differentiated cells becomes a fundamental question in biology with important implications for regenerative medicine. Although progenitor cells are important for regeneration in many organs, differentiated cells may also contribute by reverting to a proliferative state.

The terms used in this invention adhere to standard definitions generally accepted by those having ordinary skill in the art. In case any further explanation might be needed to more clearly understand the invention, some terms have been further elucidated below.

The terms “cardiomyocyte” and “heart muscle cell” are used interchangeably to refer to a cardiac muscle fiber or myocyte in the heart. The cells that comprise cardiac muscle are sometimes seen as an intermediate between skeletal and smooth muscle cells in terms of appearance, structure, metabolism, excitation-coupling and mechanism of contraction. Cardiac muscle bundles share similarities with skeletal muscle bundles with regard to the striated appearance and contraction, with both differing significantly from smooth muscle cells.

The term “regeneration” as used herein refers to the restoration of function to a lost or damaged cell, tissue or organ where function has been compromised. Regeneration capacity can be measured as a function of the cell, tissue or organ. Such functions can be, but are not limited to expression of proteins, tissue remodeling, induction of angiogenesis/vasculogenesis, reduction in hypertrophy and coordinated function as a tissue or organ, contractility and relaxation. In some embodiments, at least 20, 30, 40, 50, 60, 70, 80, 90, 95, 98, 99 or 100% of the function of the organ is regenerated.

The terms “neuregulin,” “NRG,” “neuregulin-1” and “NRG-1” are used interchangeably to refer to proteins (NP_{—}039250; SEQ ID NO:1), polypeptides, active derivatives and fragments thereof that can bind and activate ErbB3 or ErbB4 protein kinases, such as all neuregulin-1 isoforms, neuregulin EGF-like domain alone (SEQ ID NO:2), neuregulin mutants, biologically active analogs of neuregulin, and any kind of neuregulin-like gene products that also activate the above receptors. Specific fragments can comprise 100%, 95%, 90%, 85%, 80%, 75%, 70%, 50%, 40% or 30% of neuregulin or SEQ ID NO:1. Fragments can also comprise an amino-terminal or a carboxy-terminal portion of neuregulin. In preferred embodiments, neuregulin comprises a 245 residue protein comprising amino acids 1-245 (SEQ ID NO:3). In one embodiment, neuregulin comprises at least a neuregulin EGF-like domain alone (SEQ ID NO:2). In another embodiment, neuregulin comprises a 245 amino acid protein comprising amino acids 2-246 (SEQ ID NO:4). In yet another embodiment, neuregulin comprises a 71 residue protein comprising amino acids 176-246 (SEQ ID NO:5). Another embodiment, neuregulin comprises a 61 residue protein comprising amino acids 177-237 (SEQ ID NO:6). The neuregulin can also comprise 100%, 95%, 90%, 85%, 80%, 75%, 70%, 50%, 40% or 30% of SEQ ID NO:1, SEQ ID NO:2, SEQ ID NO:3, SEQ ID NO:4, SEQ ID NO:5 or SEQ ID NO:6. The neuregulin can also comprise SEQ ID NO:1, SEQ ID NO:2, SEQ ID NO:3, SEQ ID NO:4, SEQ ID NO:5 or SEQ ID NO:6 with sequences flanking either the amino-terminal or a carboxy-terminal or both terminus.

Family members of neuregulin comprise neuregulin-1 (NRG-1), neuregulin-2 (NRG-2), neuregulin-3 (NRG-3), and neuregulin-4 (NRG-4). Neuregulin is also known as heregulin, neu differentiation factor, glial growth factor, acetylcholine receptor-inducing activity, and sensory and motor neuron-derived factor. Neuregulin also comprises variants or functional homologues with conservative amino acid substitutions that do not substantially alter their biological activity. Suitable conservative substitutions of amino acids are known to those of skill in this art and may be made generally without altering the biological activity of the resulting molecule. The invention may also utilize a functional variant that is a mutant, variant, or derivative of one of SEQ ID NO:1, SEQ ID NO:2, SEQ ID NO:3, SEQ ID NO:4, SEQ ID NO:5 or SEQ ID NO:6. A variant sequence may also differ by an alteration of one or more of an addition, an insertion, a deletion and a substitution of one or more amino acids of a particular sequence. The variant sequence may comprise an amino acid sequence which differs by one or more amino acid residues from the amino acid sequences shown in a SEQ ID NO: of the invention (e.g., SEQ ID NO:1, SEQ ID NO:2, SEQ ID NO:3, SEQ ID NO:4, SEQ ID NO:5 or SEQ ID NO:6). Variant sequences may show greater than 60% homology with a coding sequence shown in a SEQ ID NO: of the invention, greater than about 70% homology, greater than about 75% homology, greater than about 80% homology, greater than about 85% homology, greater than about 90% homology or greater than about 95% homology. Those of skill in this art can recognize that, in general, single amino acid substitutions in non-essential regions of a polypeptide do not substantially alter biological activity.

On the other hand, changes to a nucleotide sequence corresponding to the amino acid sequences may result in an amino acid change at the protein level, or not, as determined by the genetic code. Nucleic acid encoding a polypeptide which is an amino acid sequence mutant, variant, or derivative of a SEQ ID NO: of the invention is further provided by the present invention.

NRG1 can act as an agonist for receptor tyrosine kinases of the epidermal growth factor receptor family, consisting of ErbB1, -2, -3, and -4 ((Fuller, S. J., Sivarajah, K., and Sugden, P. H. (2008). NRG ligands share an epidermal growth factor-like (EGF-like) domain, which is both necessary and sufficient for binding to and activating ErbB receptors. The EGF-like domain (SEQ ID NO:2) of NRG1 ligands has been shown to be structurally highly homologous to EGF. NRG1 and NRG2 ligands bind to both ErbB3 and ErbB4, whereas NRG3 and NRG4 only bind to and activate ErbB4.

More than 15 NRG1 isoforms, which result from alternative splicing of a single gene, have been identified. These isoforms can be divided into three types (I, II, or III), based on their N-terminal segments. NRG1 ligands of type I (heregulin; Neu differentiation factor; acetylcholine receptor-inducing activity (ARIA)) contain an Ig-like domain and a glycosylation-rich segment. Type II isoforms (glial growth factor) also contain an Ig-like domain but lack the glycosylation-rich segment. Type III isoforms (sensory and motor neuron-derived factor) lack both the Ig-like domain and glycosylation-rich segment but contain a cysteine-rich domain of a size comparable with the Ig-like domains of type I and II NRG1s. Variations in the C-terminal portion of the EGF-like domain of NRG1 differentiate subtypes (α, β1, β2, β3) and convey preferential binding to either ErbB3 or ErbB4. All data presented here use recombinant and nonglycosylated NRG1-β1 with or without N-terminal domains. This subtype is known to bind preferentially to ErbB3.

NRG1 isoforms are either generated from short transcripts leading to directly secreted ligands or are synthesized as transmembrane precursor proteins The membrane-bound precursors undergo cleavage between the EGF-like domain and the transmembrane domain. The result is a soluble NRG1 ligand containing both the N-terminal segments and the EGF-like domain, equivalent to NRG1 ligands obtained by direct secretion. However, direct activation of cells through cell-cell contacts between receptor-expressing cells and cells expressing membrane-bound NRG1 has also been demonstrated.

The EGF-like domain of NRG-1 has been reported to be sufficient for the basic activation of ErbB2/ErbB3 heterodimers. Furthermore, the similarity of NRG1¹⁷⁶ to EGF in terms of size and structure underscores the structural and functional similarities between their target receptors, EGFR, ErbB3, and ErbB4. As a result, most studies involving NRG1 have been carried out using NRG1¹⁷⁶ or comparable peptide ligands. However, the N-terminal segments of NRG1 are consistently retained in all isoforms in vivo, with the exception of a small fraction of NRG1 type III, which undergoes an additional cleavage event, leaving an N-terminal portion of reduced size. This suggests that the retention of the N-terminal segments of NRG1 may reflect a functional conservation despite wide variability of these N-terminal domains on the primary sequence level. One example of a functional benefit conferred by the N-terminal Ig-like domain has been reported for NRG1-β1 stimulation of acetylcholine receptor transcription in myotubes. In this case, the ability of the Ig-like domain to bind heparan sulfates facilitates the enrichment of ligand on the cell surface, resulting in an enhanced growth stimulation response at low ligand concentrations.

The ErbB family of receptor tyrosine kinases is involved in a broad spectrum of growth control and cell differentiation events. Members of this receptor family in humans include the epidermal growth factor receptor (EGFR, ErbB1), ErbB2 (HER2/Neu), ErbB3 (HER3), and ErbB4 (HER4). ErbB receptors, their ligands, and the consequences of their activation and inhibition in the myocardium are reviewed by Fuller et al. in J Mol Cell Cardiol 44, 831-854. Binding of NRG1 to ErbB4 increases its kinase activity and leads to heterodimerization with ErbB2 or homodimerization with ErbB4 and stimulation of intracellular signal transduction pathways. Mice with germline knock-out of the NRG1, ErbB2, or ErbB4 genes have thinner myocardium and die at midgestation, indicating that each of these genes is independently required for fetal cardiomyocyte generation (Gassmann, M., Casagranda, F., Orioli, D., Simon, H., Lai, C., Klein, R., and Lemke, G. (1995). Nature 378, 390-394; Meyer, D., and Birchmeier, C. (1995). Nature 378, 386-390).

The NRG1 receptor subunits ErbB2 and ErbB4 are also expressed in differentiated cardiomyocytes. It became apparent that the NRG1/ErbB2/ErbB4 signalling complex is functionally active in differentiated cardiomyocytes when women receiving breast cancer treatment with the ErbB2-blocking antibody, Herceptin, developed cardiomyopathy (Keefe, D. L. (2002). Cancer 95, 1592-1600). It has become increasingly apparent that the interaction of NRG1 and ErbB4 is important for pleiotropic effects of NRG1 that depend on the tissue context. In vitro studies have suggested that the NRG1/ErbB2/ErbB4 complex controls cardiomyocyte survival and myofibril disarray. However, these effects were not observed in knock-out mice in vivo, indicating that ErbB2 and ErbB4 may act through other cellular mechanisms.

The terms “functional” or “bioactive,” as used interchangeably herein, refer to mean a NRG-derived peptide having a non-amino acid chemical structure that mimics the structure of NRG or a NRG-derived peptide and retains the bioactivity and function of NRG in cellular and animal models. The function may comprise an improved desired activity or a decreased undesirable activity. Such a mimetic generally is characterized as exhibiting similar physical characteristics such as size, charge or hydrophobicity in the same spatial arrangement found in NRG or the NRG-derived peptide counterpart. A specific example of a peptide mimetic is a compound in which the amide bond between one or more of the amino acids is replaced, for example, by a carbon-carbon bond or other bond well known in the art (see, for example, Sawyer, Peptide Based Drug Design, ACS, Washington (1995), which is incorporated herein by reference). Non-limiting tests for a functional NRG are disclosed below. In one embodiment, the NRG-1 is capable of activating ErbB4. In another embodiment, the NRG-1 is capable of inducing heterodimerization of ErbB4 and ErbB2 receptors. The peptides of the present invention are intended to be functional in at least one bioactivity assay. Tests for functionality are described below.

The term “portion” or “fragment” as used herein refers to an amino acid sequence of the neuregulin genes that has fewer amino acids than the entire sequence of the neuregulin gene. For example, a neuregulin fragment can comprise ErbB4 receptor binding domain. In one embodiment, the neuregulin comprises at least an epidermal growth factor-like domain of NRG-1 (SEQ ID NO:2). In another embodiment, the neuregulin comprises a fragment or portion of neuregulin that includes the ErbB4 receptor binding domain to facilitate the binding of the protein fragment. For example, a neuregulin fragment comprising ErbB4 receptor binding domain can include 10%, 20%, 30%, 40% 50%, 60%, 70%, 80%, 90%, 95%, 96%, 97%, 98%, or 99% etc. of the amino acids of SEQ ID NO:1.

“Variant” as the term is used herein, is a nucleic acid or protein that differs from a reference nucleic acid or protein (i.e. a neuregulin protein or fragment thereof consistent with embodiments of the present invention), but retains essential properties (i.e., biological activity). A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. Generally, differences are limited so that the sequences of the reference polypeptide and the variant are closely similar overall and, in many regions, identical.

A variant and reference protein may differ in amino acid sequence by one or more substitutions, additions, and deletions in any combination. A substituted or inserted amino acid residue may or may not be one encoded by the genetic code. A variant of a protein may be naturally occurring such as an allelic variant, or it may be a variant that is not known to occur naturally. Non-naturally occurring variants of polynucleotides and polypeptides may be made by mutagenesis techniques or by direct synthesis. For instance, a conservative amino acid substitution may be made with respect to the amino acid sequence encoding the polypeptide. Variants can also comprise modifications to the nucleic acid or protein sequence that facilitate the function of the protein. Examples of such can include, but are not limited to, modifications of a neuregulin protein or fragment thereof to facilitate dimerization or heterodimerization.

Variant proteins encompassed by the present application are biologically active, that is they continue to possess the desired biological activity of the native protein, as described herein. The term “variant” includes any polypeptide having an amino acid residue sequence substantially identical to a sequence specifically shown herein in which one or more residues have been conservatively substituted with a functionally similar residue, and which displays the ability to mimic the biological activity of neuregulin, such as for example, activating ErbB4, and/or increasing proliferation of cardiomyocytes.

The invention may also utilize a “functional variant” that is a mutant, variant, or derivative of one of SEQ ID NO:1, SEQ ID NO:2, SEQ ID NO:3, SEQ ID NO:4, SEQ ID NO:5 or SEQ ID NO:6 that retains a biological activity of the wildtype sequence. A functional variant may differ by an alteration of one or more of an addition, an insertion, a deletion and a substitution of one or more amino acids of a particular sequence. The functional variant may comprise an amino acid sequence which differs by one or more amino acid residues from the amino acid sequences shown in a SEQ ID NO: of the invention (e.g., SEQ ID NO:1, SEQ ID NO:2, SEQ ID NO:3, SEQ ID NO:4, SEQ ID NO:5 or SEQ ID NO:6). Functional variants may show greater than 60% homology with a coding sequence shown in a SEQ ID NO: of the invention, greater than about 70% homology, greater than about 75% homology, greater than about 80% homology, greater than about 85% homology, greater than about 90% homology or greater than about 95% homology. Those of skill in this art can recognize that, in general, single amino acid substitutions in non-essential regions of a polypeptide do not substantially alter biological activity.

“Biological activity,” as used herein refers to the ability of the protein to increase DNA synthesis in cardiomyocytes, as can be tested by methods known to one skilled in the art, such as, but not limited to, BrdU uptake assay. Variants may result from, for example, genetic polymorphism or from human manipulation. Biologically active variants of a neuregulin protein of the invention will have at least about 70%, 75%, 80%, 85%, 90%, 91%, 92%, 93%, 94%, 95%, 96%, 97%, 98%, 99% or more sequence identity to the amino acid sequence for the human neuregulin protein as determined by sequence alignment programs and parameters described elsewhere herein. A biologically active variant of a protein consistent with an embodiment of the invention may differ from that protein by as few as 1-15 amino acid residues, as few as 1-10, such as 6-10, as few as 5, as few as 4, 3, 2, or even 1 amino acid residue.

### Molecular Mechanisms of Cardiomyocyte Proliferation

Differentiated cardiomyocytes coordinate contractions to perform the pumping function of the human heart. Loss of cardiomyocytes or heart muscle cells, such as after myocardial infarction, typically results in heart failure. Since only a small proportion of cardiomyocytes in adult hearts are capable of proliferation, heart transplantation remains the top choice for biological myocardial replacement therapy. Further supporting transplantation as the primary means for therapy, it has been found that only minute proliferation increases occur of approximately 0.004% in cardiomyocytes in the region bordering a myocardial infarction. Unfortunately, this proliferative rate is not sufficient for myocardial regeneration.

Stem and progenitor cells can contribute to maintenance of the cardiomyocyte number in the adult mammalian heart. Although the stem cell population can maintain the balance between cardiomyocyte death and renewal, it is insufficient to mount a significant regenerative response after injury. Transplantation of bone marrow stem cells has variable effects on cardiac function in humans. Furthermore, regenerated myocardium derived from transplanted cells has been difficult to detect in vivo. Promoting proliferation of endogenous cardiomyocytes provides an attractive and directed approach to regenerate the affected myocardium.

In contrast to adult cardiomyocytes, fetal cardiomyocytes do proliferate. After birth, cardiomyocytes binucleate, down-regulate cell cycle activators (e.g. cyclin A), up-regulate cell cycle inhibitors (e.g. retinoblastoma protein, Rb), and withdraw from the cell cycle, establishing a distinct population of nonproliferative, mature cardiomyocytes. While modifications of intrinsic cell cycle regulators can increase cell cycle activity of differentiated cardiomyocytes, extrinsic factors inducing cardiomyocyte proliferation are unknown.

Stimulation of cell cycle activity by extracellular NRG1 suggests that non-cell autonomous mechanisms control the reversion of differentiated cardiomyocytes into a proliferative state. A similar mechanism operates in differentiated tracheal cells, which are induced to proliferate by FGF signalling during Drosophila metamorphosis (Guha, A., Lin, L., and Kornberg, T. B. (2008). Proc Natl Acad Sci USA 105, 10832-10836; Weaver, M., and Krasnow, M. A. (2008). Science 321, 1496-1499). The use of neuregulin is a novel addition to other molecular strategies used to augment mammalian heart regeneration, such as the administration of recombinant periostin peptide (Kuhn, B., Del Monte, F., Hajjar, R. J., Chang, Y. S., Lebeche, D., Arab, S., and Keating, M. T. (2007). Nat Med 13, 962-969) and FGF-administration with inhibition of p38 mitogen-activated kinase (Engel, F. B., Schebesta, M., Duong, M. T., Lu, G., Ren, S., Madwed, J. B., Jiang, H., Wang, Y., and Keating, M. T. (2005). Genes Dev 19, 1175-1187; Engel, F. B., Hsieh, P. C., Lee, R. T., and Keating, M. T. (2006). Proc Natl Acad Sci USA 103, 15546-15551).

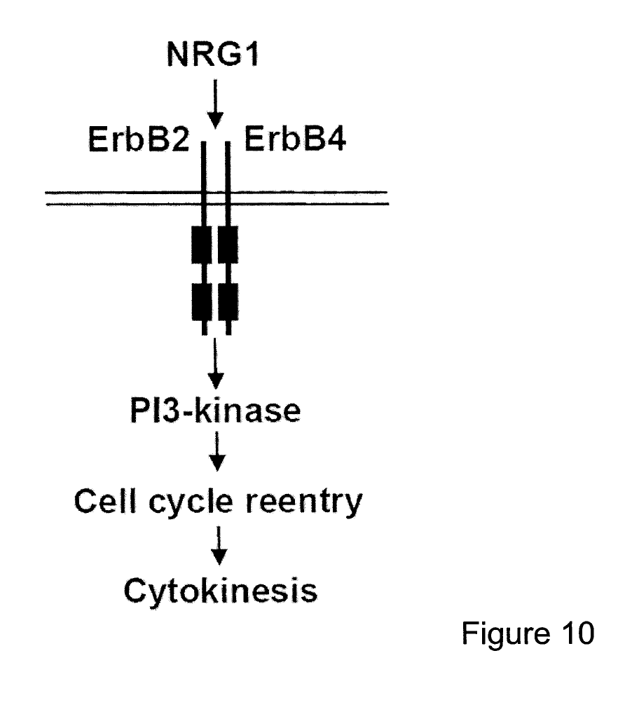

Interestingly, all four extracellular factors known to induce proliferation of differentiated cardiomyocytes, i.e. IGF1, FGF1, periostin, and NRG1, involve PI3-kinase (FIG. 10). Of note, IGF1 and FGFs also induce cardiac hypertrophy, whereas NRG1 has not been shown to display such effects. Previously, cell cycle activators have been expressed in cardiomyocytes, for example simian virus 40 large T antigen, cyclin A2, and cyclin D2 (Chaudhry, H. W., Dashoush, N. H., Tang, H., Zhang, L., Wang, X., Wu, E. X., and Wolgemuth, D. J. (2004). J Biol Chem 279, 35858-35866), resulting in increased cardiomyocyte proliferation.

### Cellular Mechanisms of Cardiomyocyte Proliferation

The Examples demonstrate that NRG1 can induce differentiated cardiomyocytes to divide over a period of at least 9 days. In one embodiment, at least about 0.1% of cardiomyocytes or heart muscle cells are induced to divide. In another embodiment, at least about 0.2%, 0.3%, 0.4%, 0.5%, 0.6%, 0.7%, 0.8%, 0.9%, 1%, 5%, 10% and 15% of cardiomyocytes or heart muscle cells are induced to divide. In comparison, in newts, lower vertebrates that regenerate their hearts, 29% of cardiomyocytes have proliferative capacity (Bettencourt-Dias, M., Mittnacht, S., and Brockes, J. P. (2003). J Cell Sci 116, 4001-4009). Zebrafish, also capable of cardiac regeneration, have more than 95% mononucleated cardiomyocytes with proliferative potential (Wills, A. A., Holdway, J. E., Major, R. J., and Poss, K. D. (2008). Development 135, 183-192). Thus, the higher regenerative capacity of adult newt and zebrafish hearts may be related to the higher prevalence of proliferation-competent mononucleated cardiomyocytes in these species.

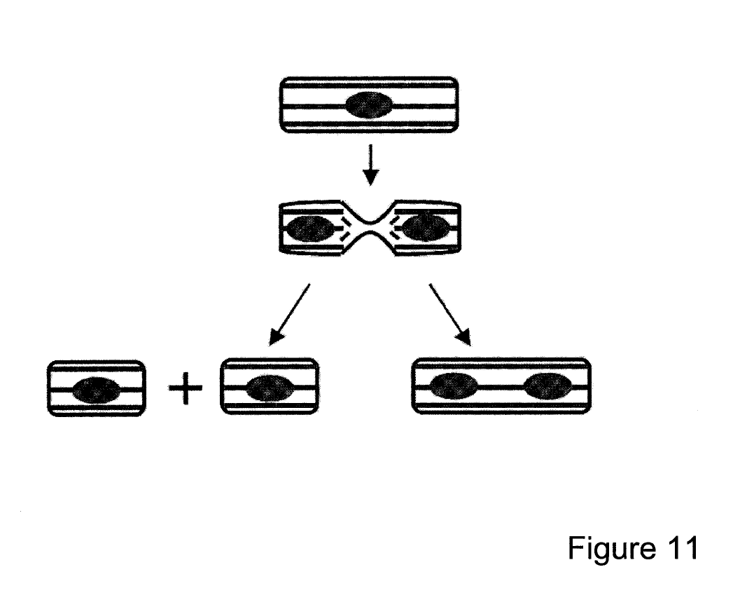

Mononucleated cardiomyocytes can have a higher proliferative potential than binucleated cardiomyocytes. Mononucleated, but not binucleated, cardiomyocytes can complete cytokinesis. However, not all mononucleated cardiomyocytes that perform karyokinesis go on to divide. The Examples demonstrate that approximately 50% of mononucleated cardiomyocytes that reentered the cell cycle, completed cyctokinesis. The other 50% did not and became binucleated (FIG. 11). One embodiment of the invention is directed to stimulating division of the heart muscle cells by inducing the heart muscle cells to reenter the cell cycle. Another embodiment is directed to about 10%, 20%, 30%, 40%, 50%, 60%, 70%, 80% and 90% of the mononucleated cardiomyocytes or heart muscle cells reenter the cell cyle. In yet another embodiment, stimulating the division of heart muscle cells further comprises increasing DNA synthesis. The invention also comprises inducing cytokinesis in the heart muscle cells. Preferrably, about 10%, 20%, 30%, 40%, 50%, 60%, 70%, 80% and 90% of mononucleated cardiomyocytes or heart muscle cells can complete cytokinesis. Although the factors that control the decision whether a mononucleated cardiomyocyte divides or becomes binucleated are unknown, this mechanism maintains the pool of proliferation-competent mononucleated cardiomyocytes, while generating terminally differentiated binucleated cardiomyocytes.

Proposals have been made in the past century that differentiated cardiomyocytes may be capable of reentering the cell cycle (Soonpaa, M. H., and Field, L. J. (1998). Circ Res 83, 15-26). However, it was concluded that the presence of differentiated sarcomeres is incompatible with cytokinesis. It was proposed that if cardiomyocytes can divide, they must possess mechanisms coordinating the cellular changes required for karyokinesis and cytokinesis with the presence of sarcomeres. One embodiment of the invention is directed to differentiated cardiomyocytes disassembling their sarcomeres in the midzone during karyokinesis and cytokinesis. Thus, the presence of the differentiated cardiomyocyte contractile apparatus does not appear to prohibit karyokinesis or cytokinesis.

NRG1 has been shown to induce differentiation of embryonic stem cells into cardiomyocytes and NRG1, ErbB2, and ErbB4-deficient mice lack myocardial trabeculations (Lee, K. F., Simon, H., Chen, H., Bates, B., Hung, M. C., and Hauser, C. (1995). Nature 378, 394-398), suggesting that NRG1 and its receptors may control cardiomyocyte differentiation during development.

### NRG1-Induced Cardiomyocyte Proliferation

Although control and NRG1-treated hearts had the same heart weight 15 weeks after myocardial infarction, NRG1-treated hearts had less hypertrophy at the cardiomyocyte level, as determined by cross-sectional area. This finding suggests that sustained cardiomyocyte replacement may have attenuated the hypertrophic drive after myocardial infarction, resulting in improved ventricular remodeling.

In one embodiment, the invention is directed to recombinant neuregulin, and biologically active fragments delivered through the cardiac extracellular matrix, to increase cardiomyocyte proliferation. In another embodiment, a method of repairing heart tissue is disclosed. The method comprises identifying a subject in need of heart tissue repair, administering to the subject an effective amount of neuregulin-1 (NRG-1), in an amount and regime effective to stimulate division of post-mitotic cardiomyocytes, and inducing proliferation of the cardiomyocytes to thereby repair heart tissue. In yet another embodiment, the subject has experienced at least one myocardial ischemia, hypoxia, stroke, and/or myocardial infarction. Another embodiment of the invention is directed to the subject having chronic ischemic heart disease.

Neuregulin can induce cell cycle re-entry of differentiated mononucleated cardiomyocytes. After experimental myocardial infarction, neuregulin can induce cardiomyocyte cell cycle re-entry, reduction in infarct size and fibrosis, and improvement in cardiac function. The application of neuregulin, and biologically active variants and fragments thereof, can enhance the regenerative capacity of adult mammalian hearts. Thus in one embodiment of the invention, administering neuregulin to a subject replaces damaged heart tissue with proliferating cardiomyocytes. In another embodiment, administration of neuregulin improves myocardial function in the subject and/or reduces myocardial hypertrophy.

### Uses of the Inventions

The invention is also applicable to tissue engineering where cells can be induced to proliferate by treatment with neuregulin, variants or fragments thereof (or such compositions together with growth factors) ex vivo. Following such treatment, the resulting tissue can be used for implantation or transplantation.

For example, in some embodiments, neuregulin, or biologically active variants or fragments thereof, are used as reagents in ex vivo applications. For example, neuregulin fragments are introduced into tissue or cells that are to be transplanted into a subject for therapeutic effect. The cells and/or tissue can be derived from an organism or subject that later receives the explant, or can be derived from another organism or subject prior to transplantation. The neuregulin compositions can be used to modulate the signaling pathway in the cells (i.e., cardiomyocytes), such that the cells or tissue obtain a desired phenotype or are able to perform a function (i.e., cardiomyocyte proliferation) when transplanted in vivo. In one embodiment, certain target cells from a patient are extracted. These extracted cells are contacted with neuregulin compositions and seeded onto biodegradable scaffolds. The cells are then reintroduced back into the same patient or other patients. Non-limiting examples of ex vivo applications include use in organ/tissue transplant, tissue grafting, or treatment of heart disease. Such ex vivo applications can also used to treat conditions associated with coronary and peripheral bypass graft failure, for example, such methods can be used in conjunction with peripheral vascular bypass graft surgery and coronary artery bypass graft surgery.

The compositions and methods of this invention have utility in research and drug development, as well as in surgery, tissue engineering, and organ transplantation. The present invention allows neuregulin, variants or fragments thereof thereof to be delivered locally, both continuously and transiently, and systemically. The invention could be used to modify or reduce scar tissue around the heart, speed up healing, and enhance cardiac tissue generation. The methods and compositions of this invention provide the ability to successfully generate new tissue, augment organ function, and preserve the viability of impaired tissues, such as ischemic tissues. The present invention can enhance the viability of tissue.

Heart failure in humans begins with reduced myocardial contractility, which leads to reduced cardiac output. The methods and composition of the invention can be used to augment heart function. For example, the invention can be used to enhance growth of cardiomyocytes in an area of the heart that has been damaged or has become ischemic. Heart diseases include, but are not limited to angina pectoris, myocardial infarction, and chronic ischemic heart disease.

Neuregulin, variants or fragments thereof, or a combination of one or more variants or fragments thereof, can be administered as compositions by various known methods, such as by injection (direct needle injection at the delivery site, subcutaneous, intravenous, etc.), oral administration, inhalation, transdermal application, catheter infusion, biolistic injectors, particle accelerators, Gelfoam, other commercially available depot materials, osmotic pumps, oral or suppositorial solid pharmaceutical formulations, decanting or topical applications during surgery, or aerosol delivery. Depending on the route of administration, the composition can be coated with a material to protect the compound from the action of acids and other natural conditions which can inactivate the compound. The composition can further include both the neuregulin compound and another agent, such as, but not limited to, a growth factor.

For therapeutic purposes, formulations for parenteral administration can be in the form of aqueous or non-aqueous isotonic sterile injection solutions or suspensions. These solutions and suspensions can be prepared from sterile powders or granules having one or more of the carriers or diluents mentioned for use in the formulations for oral administration. The compounds can be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

To administer the composition by other than parenteral administration, the composition can be coated with, or co-administer the composition with, a material to prevent its inactivation. For example, the composition can be administered to a subject in an appropriate diluent or in an appropriate carrier such as liposomes. Pharmaceutically acceptable diluents include saline and aqueous buffer solutions. Liposomes include water-in-oil-in-water CGF emulsions as well as conventional liposomes (Strejan et al., J. Neuroimmunol. 7:27 (1984)).

The composition containing at least one neuregulin protein, variants or fragments thereof can also be administered parenterally or intraperitoneally. Dispersions can also be prepared in glycerol, liquid polyethylene glycols, and mixtures thereof and in oils. Under ordinary conditions of storage and use, these preparations can contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion. In all cases, the composition must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene gloycol, and the like), suitable mixtures thereof, and vegetable oils. The proper fluidity can be maintained, for example, by the use of a coating such as licithin, by the maintenance of the required particle size in the case of dispersion and by the use of surfactants. Prevention of the action of microorganisms can be achieved by various antibacterial and antifungal agents. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the composition containing the neuregulin molecule, variants or fragments thereof in the required amount in an appropriate solvent with one or a combination of ingredients enumerated above, as required. Generally, dispersions are prepared by incorporating the composition into a sterile vehicle which contains a basic dispersion medium and the required other ingredients from those enumerated above.

Formulation of drugs is also discussed in, for example, Hoover, John E., Remington's Pharmaceutical Sciences, (1975), Mack Publishing Co., Easton, Pennsylvania; and Liberman, H. A. and Lachman, L., (1980) Eds., Pharmaceutical Dosage Forms, Marcel Decker, New York, N.Y. The term “pharmaceutically acceptable salt” means those salts which retain the biological effectiveness and properties of the compounds used in the present invention, and which are not biologically or otherwise undesirable. Such salts may be prepared from inorganic and organic bases. Salts derived from inorganic bases include, but are not limited to, the sodium, potassium, lithium, ammonium, calcium, and magnesium salts. Salts derived from organic bases include, but are not limited to, salts of primary, secondary and tertiary amines, substituted amines including naturally-occurring substituted amines, and cyclic amines, including isopropylamine, trimethylamine, diethylamine, triethylamine, tripropylamine, ethanolamine, 2-dimethylaminoethanol, tromethamine, lysine, arginine, histidine, caffeine, procaine, hydrabamine, choline, betaine, ethylenediamine, glucosamine, N-alkylglucamines, theobromine, purines, piperazine, piperidine, and N-ethylpiperidine. It should also be understood that other carboxylic acid derivatives, for example carboxylic acid amides, including carboxamides, lower alkyl carboxamides, di(lower alkyl) carboxamides, may be used.

Neuregulin (or pharmaceutically acceptable derivatives thereof) may be administered per se or in the form of a pharmaceutical composition wherein the active compound(s) is in admixture or mixture with one or more pharmaceutically acceptable carriers, excipients or diluents. Pharmaceutical compositions may be formulated in conventional manner using one or more physiologically acceptable carriers comprising excipients and auxiliaries which facilitate processing of the active compounds into preparations which can be used pharmaceutically. Proper formulation is dependent upon the route of administration chosen.

In one aspect, the methods of the invention can be used to repair heart tissue. In one embodiment, the neuregulin composition of the present invention can be incorporated into polymers, such as those used to make cardiovascular stents, or used as a coating on stents used after angioplasty. The neuregulin composition of the present invention can, for example, be combined with and/or impregnated into polymers (e.g., biodegradable polymers, slow release polymers, and/or controllable or inducible-release polymers) such that the composition can be delivered to the target site over time. The polymer can be impregnated with one or more composition of the present invention such that release can be controlled and directed to the target area (e.g., injured tissue). In addition, the stents can comprise one of more compositions of the present invention combined with other compounds (e.g., antioxidants, periostin, and FGF) to provide synergist effects and/or with other drugs (e.g., antibiotics, growth factors, cholesterol reducing agents, such as statins, anti-neoplastics, immunosupressives, migration inhibitors, and enhanced healing factors) to repair the heart tissue.

When the composition containing the neuregulin composition is suitably protected, as described above, the composition can be orally administered, for example, with an inert diluent or an assimilable edible carrier. The composition and other ingredients can also be enclosed in a hard or soft shell gelatin capsule, compressed into tablets, or incorporated directly into the subject's diet. For oral therapeutic administration, the composition can be incorporated with excipients and used in the form of ingestible tablets, buccal tablets, troches, capsules, elixirs, suspensions, syrups, wafers, and the like. The percentage of the compositions and preparations can, of course, be varied. The amount of active compound in such therapeutically useful compositions is such that a suitable dosage will be obtained.

The tablets, troches, pills, capsules and the like can also contain a binder, an excipient, a lubricant, or a sweetening agent. Various other materials can be present as coatings or to otherwise modify the physical form of the dosage unit. For instance, tablets, pills, or capsules can be coated with shellac, sugar or both. Of course, any material used in preparing any dosage unit form should be pharmaceutically pure and substantially non-toxic in the amounts employed. As used herein “pharmaceutically acceptable carrier” includes any solvents, dispersion media, coatings, antibacterial and antifungal agents, isotonic and absorption delaying agents, and the like. The use of such media and agents for pharmaceutically active substances is well known in the art. Except insofar as any conventional media or agent is incompatible with the active compound, use thereof in compositions of the invention is contemplated.

If administered per os, the compounds can be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets can contain a controlled-release formulation as can be provided in a dispersion of active compound in hydroxypropylmethyl cellulose. In the case of capsules, tablets, and pills, the dosage forms can also comprise buffering agents such as sodium citrate, or magnesium or calcium carbonate or bicarbonate. Tablets and pills can additionally be prepared with enteric coatings.

It is especially advantageous to formulate compositions of the invention in dosage unit form for ease of administration and uniformity of dosage. Dosage unit form as used herein refers to physically discrete units suited as unitary dosages for the subjects to be treated. Each dosage contains a predetermined quantity of active compound calculated to produce the desired therapeutic effect in association with the required pharmaceutical carrier. The specification for the novel dosage unit forms of the invention is dependent on the unique characteristics of the composition containing neuregulin, variants or fragments thereof, and the particular therapeutic effect to be achieved. Dosages are determined by reference to the usual dose and manner of administration of the ingredients. In some embodiments, neuregulin is administered at 0.01 mg/kg, 0.05 mg/kg, 0.1 mg/kg, 0.5 mg/kg, 1 mg/kg, 2 mg/kg, 3 mg/kg, 4 mg/kg, 5 mg/kg, 10 mg/kg, 15 mg/kg and 20 mg/kg. In a preferred embodiment, neuregulin is administered at about 1 mg/kg. In some other embodiments of the invention, neuregulin is administered for a duration of at least 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months and 1 year.

One skilled in the art will appreciate further features and advantages of the invention based on the above-described embodiments. Accordingly, the invention is not to be limited by what has been particularly shown and described, nor by the examples set forth below. All publications and references cited herein are expressly incorporated herein by reference in their entirety.

### EXAMPLES

The Examples show that neuregulin and biologically active fragments thereof induce cell cycle re-entry of differentiated mammalian cardiomyocytes. Neuregulin stimulates mononuclear cardiomyocytes, present in the adult mammalian heart, to undergo the full mitotic cell cycle. Neuregulin activates ErbB receptors located in the cardiomyocyte cell membrane. Neuregulin-induced cardiomyocyte proliferation requires activation of the ErbB signaling pathways. NRG1 induces mononucleated, but not binucleated, cardiomyocytes to divide. In vivo, genetic inactivation of ErbB4 reduces cardiomyocyte proliferation, while increasing ErbB4 expression enhances it. Injecting NRG1 in adult mice induces cardiomyocyte cell cycle activity and promotes myocardial regeneration, leading to improved function after myocardial infarction. Undifferentiated progenitor cells did not contribute to NRG1-induced cardiomyocyte proliferation. Neuregulin and the pathway it regulates provides a new target for innovative strategies to treat heart failure.

### Example 1

### Materials and Methods

### Cell Cycle Activity In Vitro

Experiments were approved by the Animal Care and Use Committee. Ventricular cardiomyocytes were isolated from male Wistar rats (12 week old, 300 g, Charles River Laboratories). We added NRG1 (EGF-like domain, amino acids 176-246 (SEQ ID NO:5); 100 ng/mL, R&D Systems), the peptide consisting of the four fasciclin 1 domains of human periostin (500 ng/mL; BioVendor) (SEQ ID NO:7), FGF1 (100 ng/mL; R&D Systems), HB-EGF (10 ng/mL; R&D Systems), or PDGF-BB (10 ng/mL; Peprotech). For detection of DNA synthesis, we added BrdU (30 μM) for the last three days. The c-ErbB2/Neu blocking antibody was from Calbiochem.

### Mouse Strains

ErbB4F/F mice were obtained from the NIH-sponsored Mutant Mouse Repository at University of California Davis and were originally produced by Dr. Kent Lloyd (Golub et al., 2004). The α-MHC-MerCreMer mice were obtained from the Jackson Laboratories and originally generated by Dr. Jeffrey Molkentin (Sohal et al., 2001). The α-MCH-ErbB4 mice, originally generated by Dr. Martin Gassmann (Tidcombe et al., 2003), were obtained from Dr. Gabriel Corfas (Children's Hospital Boston). The Rosa26lacZ mice were obtained from Jackson Laboratories and originally produced by Dr. Phillippe Soriano (Soriano, 1999). All mice were crossed to C57B1/6 mice purchased from Taconic Laboratories. The Children's Hospital Institutional Animal Care and Use Committee approved all of the animal experiments.

### Determination of Recombination

To determine deletion of ErbB4 exon 2 at the genomic level, we performed PCR on genomic DNA prepared from myocardium as described (Jackson-Fisher et al., 2006). Tamoxifen-injected, but not oil-injected α-MHC-MerCreMer+/+; ErbB4_{F/F} mice had a 507 by PCR product indicating high specificity of the deletion system. Expression of ErbB4 protein, detected with an antibody provided by Cary Lai (#0616, Salk Institute) and visualized by immunofluorescence microscopy, was decreased in cardiomyocytes isolated from test mice. To determine the efficiency of our deletion protocol, we crossed the α-MHC-MerCreMer allele into a Rosa26lacZ background (Soriano, 1999) and quantified the number of (3-galactosidase-positive cardiomyocytes after injection of tamoxifen. We detected β-galactosidase activity in 83.5±5.5% of cardiomyocytes (n=3) dispersed throughout the myocardium and no β-galactosidase activity in the absence of Cre. In conclusion, our inducible deletion strategy was very efficient and highly specific for differentiated cardiomyocytes.

### Determination of Cardiomyocyte Volume

We determined the volume of isolated cardiomyocytes with morphometry. We visualized the contractile apparatus of isolated cardiomyocytes with immunofluorescence microscopy and acquired stacks of 0.25 μm spaced confocal slices. We determined the cardiomyocyte boundary by thresholding the confocal channel visualizing the contractile apparatus to (mean fluorescence intensity+2 standard deviations) and subtracted the nuclear volume. We used the volume volume analysis tool in Slidebook software to determine the cardiomyocyte volume.

### Clonal Analysis

We treated α-MHC-MerCreMer+/+; Rosa26R+/− mice with tamoxifen (10 μg/gm i.p.×1) at postnatal age 15 days to induce site-specific recombination, leading to sparse labeling of differentiated cardiomyocytes. Five days later, NRG1 injections were given with simultaneous labeling with BrdU (1 mg/mL drinking water). We euthanized mice after 9 days and prepared 14 μm cryosections. After fixation in 70% ethanol for 15 min, we developed X-gal staining by incubating in 1 mg/mL 5-bromo-4-chloro-3-indolyl-β-D-galactopyranoside for 12-48 hr. We performed clonal analysis by quantifying at least 200-400 X-gal positive cardiomyocytes clusters per heart.

### Stem Cells Contribution Analysis

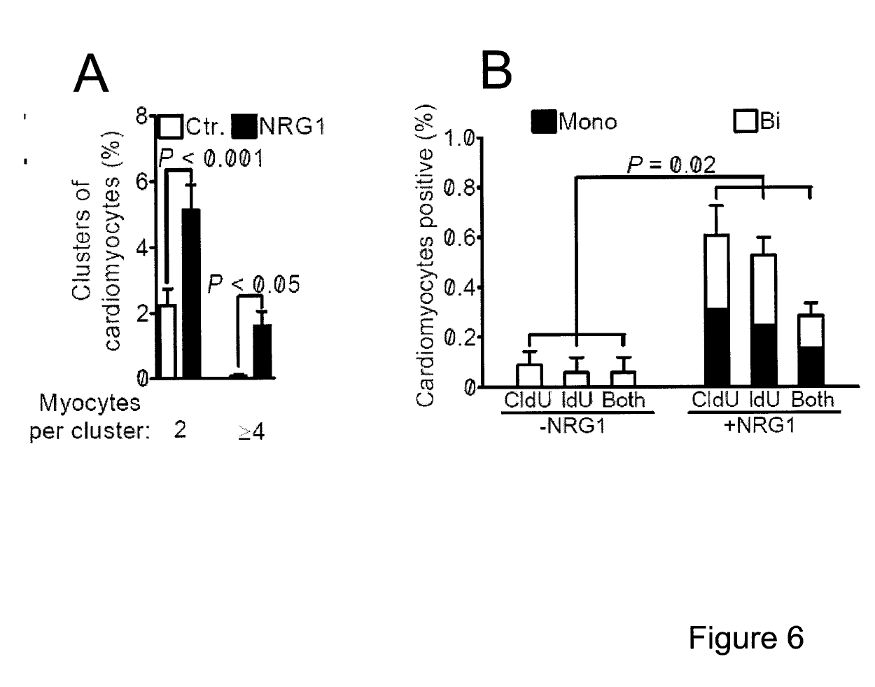

Using tamoxifen-induced site-specific recombination in the α-MHC-MerCreMer+/−; Rosa26R+/− strain, we labeled approximately 80% of differentiated cardiomyocytes with a permanent genetic tag. Cell cycle activity in genetically unlabeled cardiomyocytes may be the result of an NRG1-effect on undifferentiated cardiac stem or progenitor cells. However, cell cycle activity in the tagged and untagged population of cardiomyocytes was the same, suggesting that undifferentiated stem or progenitor cells did not contribute to NRG1-induced cardiomyocyte cycling. Nevertheless, we considered that NRG1-induced cell cycle activity in genetically unlabeled cardiomyocytes may be the result of stem or progenitor cell proliferation. If NRG1-induced cardiomyocyte cell cycle activity originated from differentiated cardiomyocytes, the genetic labeling efficiency should not influence the proliferative rate. Accordingly, the proliferative rate should be identical in genetically labeled and unlabeled cardiomyocytes irrespective of the percentage of genetically labeled cardiomyocytes. In contrast, if the NRG1-induced cardiomyocyte cell cycle activity in the genetically unlabelled population were derived from undifferentiated stem or progenitor cells, the genetic labeling efficiency should influence the proliferative rate. Accordingly, the proliferative rate should change with the percentage of genetically labeled cardiomyocytes. To address these possibilities, we modified our protocol such that we genetically labeled decreasing proportions of differentiated cardiomyocytes. The proliferative rate was the same at low, intermediate, and high genetically labeled proportions of differentiated cardiomyocytes (FIG. 6D). Thus, the proportion of cycling cardiomyocytes was not a function of the labeling efficiency. In conclusion, NRG1-induced cardiomyocyte cell cycle activity did not originate from undifferentiated stem or progenitor cells.

### Mouse Strains, Cardiomyocyte Proliferation, and Genetic Fate Tracking In Vivo

We used the α-MHC-MerCreMer strain to delete the loxP-flanked exon 2 of ErbB4. We injected tamoxifen in mice with two alleles of α-MHC-MerCreMer and one (control) or two (test) ErbB4 alleles with floxed exon 2. Recombination was detected in 83.5±2% of cardiomyocytes (n=7). We used heterozygotes of the α-MHC-ErbB4 transgenic strain to increase ErbB4 expression. NRG1 (2.5 μg/mouse dissolved in 0.1% bovine serum albumin, i.p.) or 0.1% bovine serum albumin were injected daily for 9 days. To determine the efficiency of recombination, for genetic fate tracking experiments, to analyze clonal proliferation of cardiomyocytes, and to determine the contribution of stem cells, we crossed the α-MHC-MerCreMer allele into the Rosa26lacZ strain, injected tamoxifen, and analyzed β-galactosidase-positive and—negative cardiomyocytes. Injections of BrdU (70 μmol/kg, i.p.), with a tissue half-life of 2 h, were given every 12 hr. For continuous labelling, one injection of thymidine analog was given followed by addition to the drinking water (1 mg/mL) for 9 days. Cardiomyocytes were isolated 24 hr later by Langendorff perfusion with collagenase II (20 mg/mL, Invitrogen) and protease XIV (5 mg/mL, Sigma). Cell cycle activity and number of cardiomyocyte nuclei were determined by immunofluorescence microscopy.

### Microscopy, Cardiomyocyte Volumes and Cross-Sectional Area, and Sarcomere Disassembly

Immunofluorescence microscopy and fate tracking in vitro were performed as described. We used primary antibodies against tropomyosin (Developmental Studies Hybridoma Bank), troponin I (Santa Cruz), BrdU (Abeam), and aurora B kinase (BD Biosciences) for detection and Alexa-fluorophore-conjugated secondary antibodies (Invitrogen) for visualization. We visualized nuclei with 4′,6′-diamidino-phenylindole (DAPI, Invitrogen). The y value for image acquisition was set at one. Lookup table settings were linear (details of image acquisition in Table S7). For live cell imaging, we maintained cardiomyocytes in an environmental chamber (Tokai-HIT) fitted on the motorized stage (Prior) of an inverted Olympus IX-81 microscope. We used adenoviral transduction to express a fusion construct of histone 2B-GFP under control of the chicken troponin T promoter (cTNT-H2B-GFP). Movies were acquired with a ×20 objective, NA 0.45, by a CCD (Hamamatsu) at multiple locations in 1 hr intervals. To determine cardiomyocyte dimensions and volume, we visualized cardiac contractile apparati in isolated cardiomyocytes with an antibody against troponin I (Santa Cruz Biotechnology), acquired confocal stacks with a step size of 0.5 μm, and analyzed by histomorphometry. To determine cardiomyocyte cross-sectional area, we stained cryosections of 14 μm thickness with Masson's Trichrome and determined the area after digital thresholding (Metamorph, Molecular Devices). To determine sarcomere disassembly, we stained cryosections with either α-actinin (Sigma) or myomesin (Developmental Studies Hybridoma Bank) antibodies to visualize Z-disk or M-band, respectively. Karyokinesis was visualized with a phosphorylated histone H3 antibody (Upstate). Cytokinesis was visualized with an antibody against aurora B kinase (Sigma). Images were obtained using a spinning-disk confocal microscope (DSU, Olympus).

### Determination of Cardiomyocyte and Stem Cell Contribution

Briefly, to visualize proliferation of individual cardiomyocytes in vivo, we induced sparse genetic labeling in α-MHC-MerCreMer+/+; Rosa26R+/− mice with tamoxifen (5 μg/gm i.p.×1), injected NRG1, and quantified clusters of one, two, and more genetically labeled cardiomyocytes on X-gal stained sections. To determine the contribution of undifferentiated stem- and progenitor cells, we genetically labeled differentiated cardiomyocytes in α-MHC-MerCreMer^{+/+}; Rosa26R^{+/-} mice. If NRG1-induced cardiomyocyte cell cycle activity originated from differentiated cardiomyocytes, the proliferative rate should be identical in genetically labeled and unlabeled cardiomyocytes, irrespective of the percentage of genetically labeled cardiomyocytes. In contrast, if the NRG1-induced cardiomyocyte cell cycle activity originated from undifferentiated progenitor cells, the proliferative rate should change with the percentage of genetically labeled cardiomyocytes. We addressed these possibilities by quantifying the proliferative rate of X-gal-positive and -negative cardiomyocytes across a range of labeling efficiencies.

### Quantification of Myocardial Function and Regeneration

We performed sedated echocardiography using a VisualSonics device with a 40 MHz probe. To quantify myocardial regeneration, we analyzed ×1.5 magnification pictures of AFOG-stained cryosections. Cardiomyocyte nuclei were counted using the optical dissector method. Cardiomyocyte apoptosis was determined using the ApopTag Red In Situ apoptosis detection kit (Chemicon).

### Statistical Analyses

Observations were quantified independently from one another and in a blinded manner. Numeric data are presented as mean±s.e.m. We tested statistical significance with the t-test and analysis of variance (ANOVA). We used sigmoidal nonlinear or linear regression to fit data (GraphPad). The α-value was set at 0.05.

### Example 2

### NRG1 Stimulates Mononucleated Cardiomyocytes to Proliferate

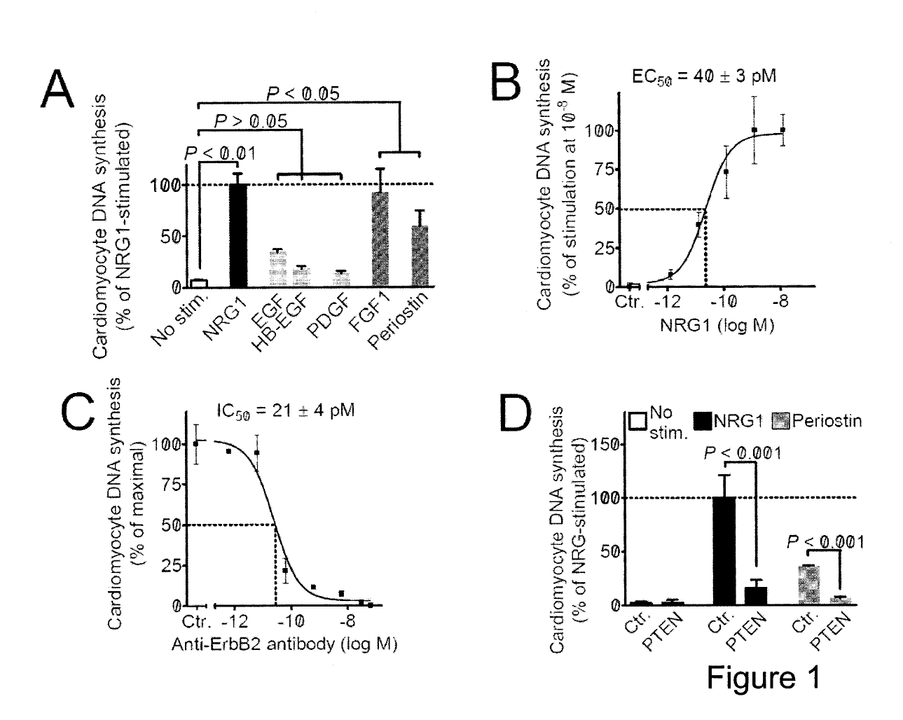

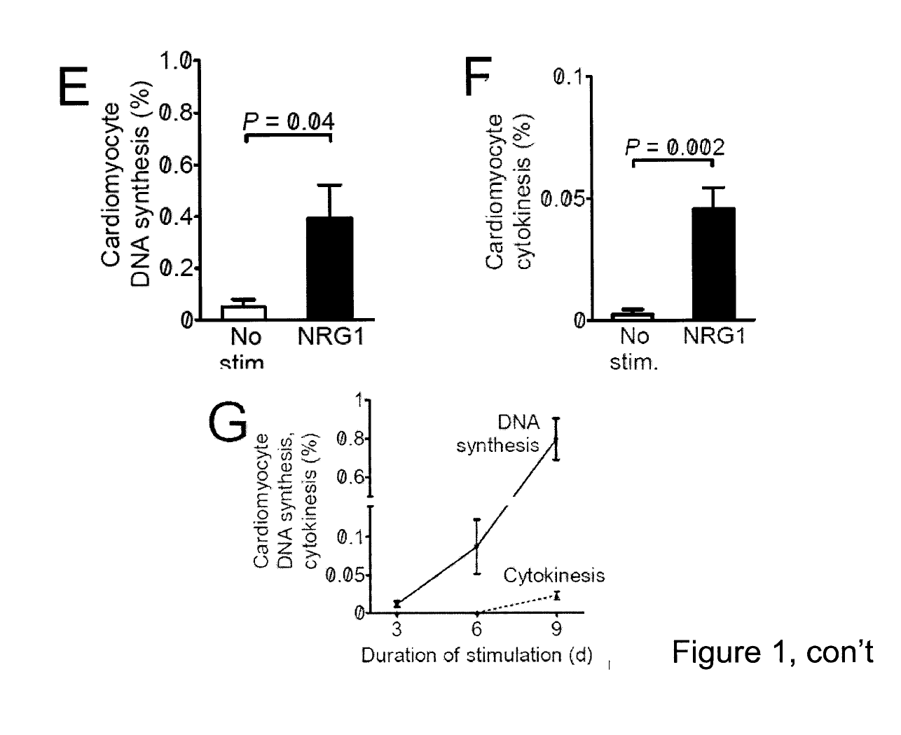

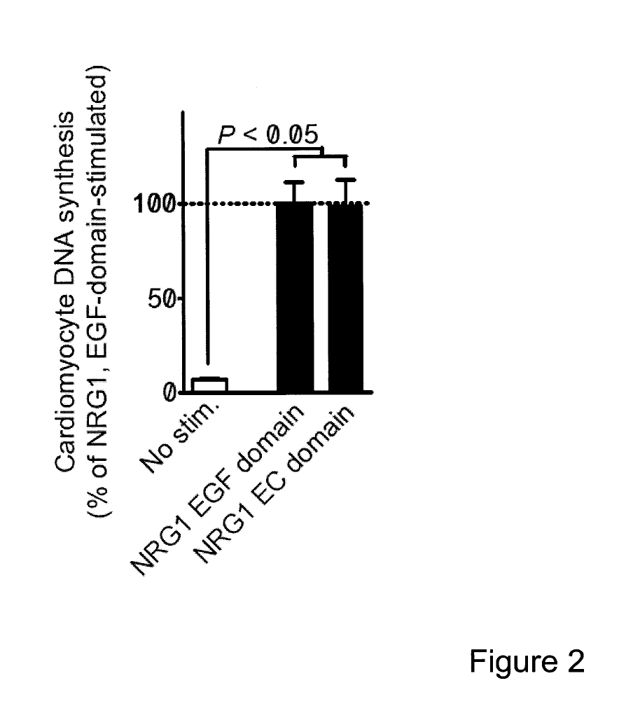

To identify factors that promote myocardial regeneration, we screened extracellular factors for their ability to induce DNA synthesis in primary adult rat ventricular cardiomyocytes. Three extracellular factors induced cardiomyocyte cell cycle reentry. Two have been previously identified: fibroblast growth factor-1 and periostin. The novel factor was the epidermal growth factor-like domain of NRG1β (FIG. 1A, FIG. 2). NRG1 induced concentration-dependent DNA synthesis, which best fit a sigmoidal function, suggesting a receptor-mediated process (FIG. 1B). The half-maximal stimulation EC₅₀ was at 40±3 pM (n=3, FIG. 1B), indicating a high-affinity interaction with the receptor. In cardiomyocytes, NRG1 binds to ErbB4, which leads to formation and activation of ErbB2/ErbB4 hetero- or ErbB4/ErbB4 homodimers. To determine whether ErbB2 is required for cardiomyocyte cell cycle reentry, we added a fixed concentration of NRG1 (125 pM) and increasing concentrations of ErbB2-blocking antibody. The anti-ErbB2 antibody inhibited NRG1-stimulated DNA synthesis with an IC₅₀ of 21±4 pM (n=3, FIG. 1C), suggesting that ErbB2 is required for the NRG1 effect.

The phosphatidylinositol-3-OH kinase (PI3-kinase) pathway is required for cardiomyocyte cell cycle reentry induced by FGF and periostin. Using functional inhibition with PTEN, we demonstrate that NRG1 also required the PI3-kinase pathway (FIG. 1D), thus suggesting that different extracellular factors induce cardiomyocyte proliferation by activating pathways that converge at PI3-kinase. In summary, the ternary complex of NRG1, ErbB2, and ErbB4 enhances cardiomyocyte cell cycle activity in a PI3-kinase-dependent mechanism.

To determine whether NRG1 induces differentiated cardiomyocytes to reenter the cell cycle, we ascertained the phenotype of individual cardiomyocytes before stimulation. NRG1 induced DNA synthesis in 0.4±0.1% of cardiomyocytes over a period of 3 days (FIG. 1E). We detected cytokinesis by visualizing aurora B kinase, a required component of the contractile ring at the site of cytoplasmic separation. In NRG1-stimulated samples, 0.05±0.01% of cardiomyocytes were in the process of cytokinesis (FIG. 1F). Because most differentiated cardiomyocytes are multinucleated, it is possible that they undergo cytokinesis without prior DNA synthesis and karyokinesis. We therefore analyzed the time course of NRG1-induced cardiomyocyte DNA synthesis and cytokinesis and found that DNA synthesis preceded cytokinesis (FIG. 1G). In addition, cardiomyocytes in cytokinesis had BrdU-positive nuclei, indicating that they underwent DNA synthesis prior to cytokinesis (FIG. 1H).

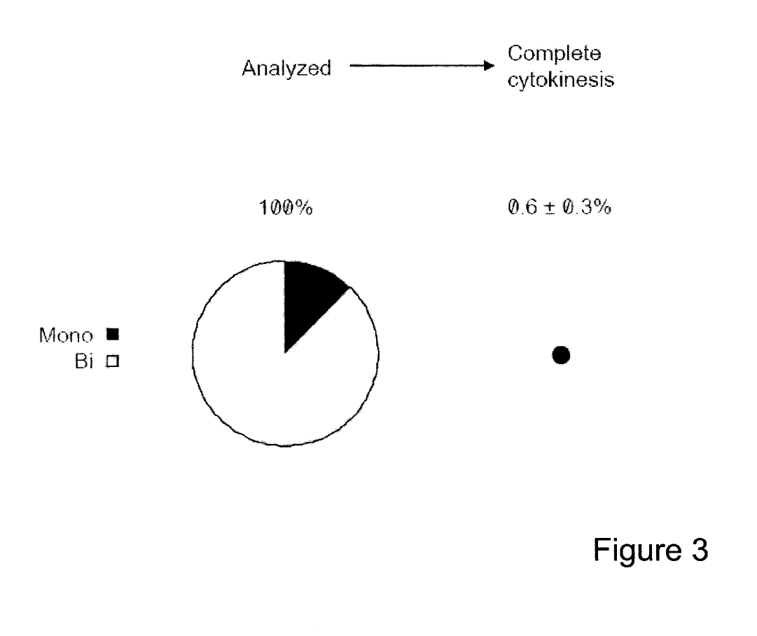

To determine whether differentiated cardiomyocytes complete cytokinesis, we used video microscopy. NRG1 induced 3±1.4% of differentiated cardiomyocytes to perform karyokinesis and 0.6±0.3% to perform cytokinesis (n=716, FIG. 3). All of the observed cycling cardiomyocytes were viable for the entire duration of observation (75.4±17.7 hr, range 15-163 hr, n=25). In summary, these data indicate that NRG1 induces differentiated cardiomyocytes to reenter the cell cycle from S-phase and to complete cytokinesis in vitro.

Using video microscopy, we prospectively determined the proliferative potential of mono- and binucleated cardiomyocytes (FIG. 1I). We found that NRG1 induced 32.6±4.8% (n=88) of mononucleated cardiomyocytes to perform karyokinesis. In contrast, only 1±0.5% of all binucleated cardiomyocytes (n=628) performed karyokinesis (P=0.02, t-test). Moreover, 45.8±20.8% (n=11) of mononucleated cardiomyocytes that entered cytokinesis, also completed cytokinesis with abscission; the rest became binucleated. In total, 0.6±0.3% of all cardiomyocytes divided, all of which were mononucleated.

### Example 3

### ErbB4 Controls Postnatal Cardiomyocyte Proliferation In Vivo

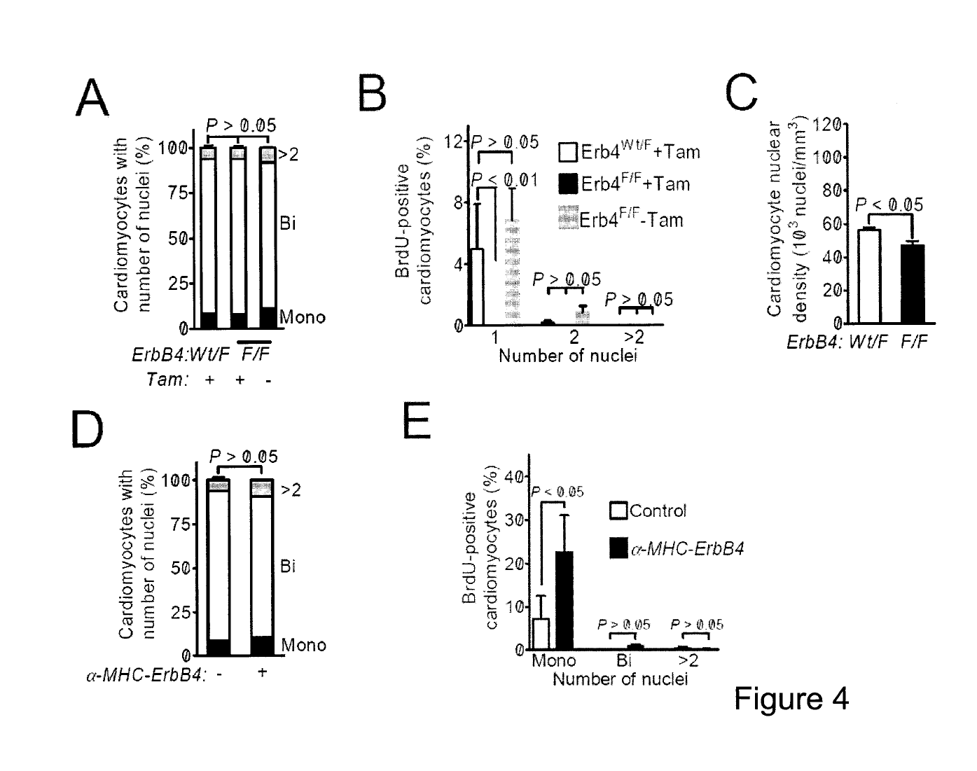

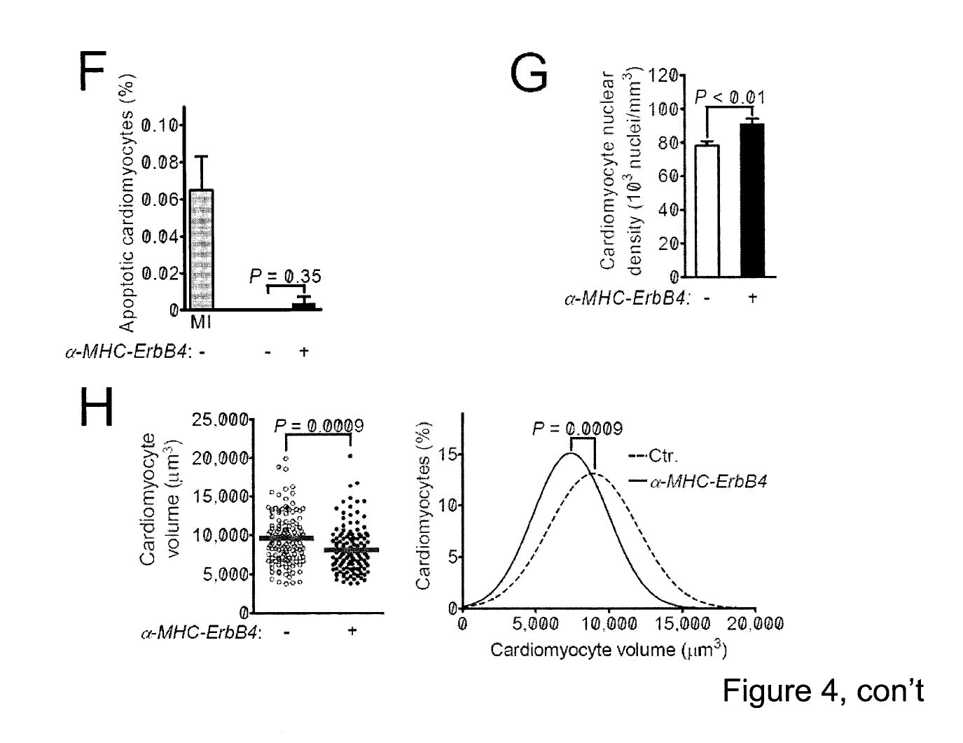

To determine whether the NRG1/ErbB2/ErbB4 complex controls cardiomyocyte proliferation in postnatal hearts in vivo, we disrupted the complex by genetically inactivating the ErbB4 gene. We treated α-MHC-MerCreMer^{+/+}; ErbB4^{F/F} mice (test) and α-MHC-MerCreMer^{+/+}; ErbB4^{Wt/F} (control littermates) with tamoxifen. Following ErbB4 inactivation on postnatal days 2-4, we analyzed the effect on postnatal day 19. Cardiomyocyte differentiation was not affected, as demonstrated by two observations: the formation of bi- and multinucleated cardiomyocytes was not altered (FIG. 4A), and cardiomyocytes from test and control mice had indistinguishable morphology (FIG. 4B). To determine whether ErbB4 is required for postnatal cardiomyocyte cell cycling, we quantified cardiomyocytes that incorporated BrdU (FIG. 4B). After 5 injections of BrdU on postnatal days 16-18, test mice had no detectable cardiomyocyte BrdU uptake, while control mice had 5±2.9% (n=4) BrdU-positive mononucleated cardiomyocytes (P<0.01, FIG. 4B). Neither α-MHC-MerCreMer^{+/+}; ErbB4^{F/F} mice in the absence of tamoxifen (FIG. 4B) nor α-MHC-MerCreMer^{+/+}; ErbB4^{F/F} mice treated with tamoxifen had reduced NRG1-induced cardiomyocyte cycling, thus confirming that inactivation of both ErbB4 alleles was required. To test whether the decrease in cell cycle activity in ErbB4-inactivated mice would result in lower cardiomyocyte numbers, we determined the volume density of cardiomyocyte nuclei, which was 20% lower (FIG. 4C, Table 1). Taken together, ErbB4 is required for normal postnatal cardiomyocyte proliferation.

TABLE 1
Characterization of effect of ErbB4 inactivation on cardiomyocyte
proliferation. Morphometric and histologic analyses were performed
on resected hearts at 19 days of age. Echocardiography was
performed with a 40 MHz probe and images were recorded and
analyzed with a Visualsonics ultrasound machine. Statistical
significance was tested by ANOVA (Bonferroni method).

| | | | Statistical |
|---|---|---|---|
| | ErbB4^{Wt/F} | ErbB4^{F/F} | significance |
| Histology: | | | |
| n | 9 | 6 | |
| Body weight (gm) | 6.8 ± 0.4 | 8.5 ± 0.6 | P > 0.05 |
| Heart weight (mg) | 49.6 ± 2.7 | 58.2 ± 4.8 | P > 0.05 |
| Heart/body weight | 7.3 ± 0.2 | 6.8 ± 0.2 | P > 0.05 |
| (mg/gm) | | | |
| Tissue density of | 58,630 ± 1,546 | 47,340 ± 2,500 | P < 0.05 |
| cardiomyocyte | | | |
| nuclei (mm [symbol] ) | | | |
| Echocardiography: | | | |
| n | 8 | 6 | |
| Contractility | 48.5 ± 3.0 | 47.9 ± 2.1 | P > 0.05 |
| (FS, %) | | | |
| Interventricular | 0.7 ± 0.04 | 0.7 ± 0.03 | P > 0.05 |
| septum in diastole | | | |
| (mm) | | | |
| Left ventricular | 2.3 ± 0.1 | 2.0 ± 0.4 | P > 0.05 |
| internal dimension | | | |
| in diastole (mm) | | | |

[symbol]

indicates data missing or illegible when filed

To determine whether ErbB4 is sufficient to increase cycling of differentiated cardiomyocytes, we overexpressed ErbB4 under control of the α-MHC promoter (α-MHC-ErbB4; which did not alter the percentage of mono- and multinucleated cardiomyocytes, indicating that ErbB4 did not affect the normal postnatal formation of multinucleated cardiomyocytes (FIG. 4D). To analyze cardiomyocyte cell cycling, we quantified cardiomyocyte BrdU uptake. Control mice at 14 days of age had BrdU uptake in 7.2±5.3% (n=6) of mononucleated cardiomyocytes (FIG. 4E). In contrast, α-MHC-ErbB4 transgenic mice had BrdU uptake in 22.6±8.5% (n=5) of mononucleated cardiomyocytes, a 3-fold increase (P<0.05). ErbB4-transgenesis did not affect cardiomyocyte apoptosis (FIG. 4F). The increased cell cycle activity in ErbB4-transgenic hearts resulted in a higher volume density of cardiomyocyte nuclei (FIG. 4G), consistent with smaller cardiomyocytes (FIG. 4H) of normal proportions. In the presence of identical myocardial mass and volume and identical proportions of mono- and multinucleated cardiomyocytes (Tables 2-3, FIG. 4D), this indicates that α-MHC-ErbB4 transgenic hearts have more cardiomyocytes that are smaller. In summary, these results demonstrate that ErbB4 is sufficient to increase cardiomyocyte proliferation in vivo.

TABLE 2
Characterization of effect on cardiomyocyte proliferation in α-MHCErbB4
transgenic mice. Morphometric and histologic analyses were performed on
resected hearts at 15 days of age. Echocardiography was performed with a
10 MHz probe and images were recorded and analyzed with a Vivid i
ultrasound machine. Statistical significance was tested by ANOVA
(Bonferroni method).

| | | α-MHC-ErbB4 | Statistical |
|---|---|---|---|
| | Control | transgene | significance |
| Histology: | | | |
| n | 5 | 6 | |
| Body weight (gm) | 7.7 ± 0.2 | 7.3 ± 0.2 | P > 0.05 |
| Heart weight (mg) | 52.4 ± 2.1 | 51.5 ± 1.6 | P > 0.05 |
| Heart/body weight | 6.8 ± 0.2 | 7 ± 0.1 | P > 0.05 |
| (mg/gm) | | | |
| Tissue density of | 75,520 ± 4,435 | 90,720 ± 3,375 | P < 0.001 |
| cardiomyocyte | | | |
| nuclei (mm [symbol] ) | | | |
| Echocardiography: | | | |
| n | 6 | 10 | |
| Contractility | 51.1 ± 3.5 | 48.6 ± 2.2 | P > 0.05 |
| (FS, %) | | | |
| Interventricular | 0.7 ± 0.1 | 0.6 ± 0.05 | P > 0.05 |
| septum in diastole | | | |
| (mm) | | | |
| Left ventricular | 2.4 ± 0.2 | 2.5 ± 0.1 | P > 0.05 |
| internal dimension | | | |
| in diastole (mm) | | | |

[symbol]

indicates data missing or illegible when filed

TABLE 3
Cardiomyocyte proliferative indices of selected vertebrates.

| | Species | | | |
|---|---|---|---|---|
| | Newt (Notophthalmus | Zebrafish (Dania rerio), in | Rat (Wistar, this report), in | Mouse (C57, this report), |
| | viridescens)¹, in vitro | vivo² | vitro | in vivo |
| Age of animals | Adult | Adult | 3 months | 2 [symbol] months |
| | | (100 mg) | (250 [symbol] 399 gm) | (20 [symbol] 25 gm) |
| Mitogen added | 10% fetal bovine serum | Endogenous | Neuregulin 1 (100 ng/mL) | Neuregulin 1 (2.5 μg/mouse |
| | | | | i.p.) |
| Mononucleated | >98% | 95.1% | 11.2% | −10% |
| fraction (%) | | | | |
| DNA synth. (%) | 75% | 10.1% | 0.4-1% | 0.9% |
| Label time | At 18 days in culure | Daily BrdU × 3 days | At 9 days in culture | 9 days continuous BrdU |
| | ³H-thymidine × 15 days | | BrdU × 3 days | |
| Karyokinesis | 1.2% (phosphorylated | ND | 3% (metaphase plate | 0.4% (instantaneous, of |
| (%) | histane H3) | | cumulative over 6 days) | mononucleated) |
| | 60% (cumulative over 18 | | | |
| | days) | | | |
| Cytokinesis | 29% (cumulative over 18 | ND | 0.6% (cumulative over 6 | 0.25% (instantaneous, of |
| (abscission, %) | days) | | days) | mononucleated) |

[symbol]

indicates data missing or illegible when filed

### Example 4

### NRG1 Induces Differentiated Cardiomyocyte Cell Cycle Re-Entry, Karyokinesis, and Cytokinesis In Vivo

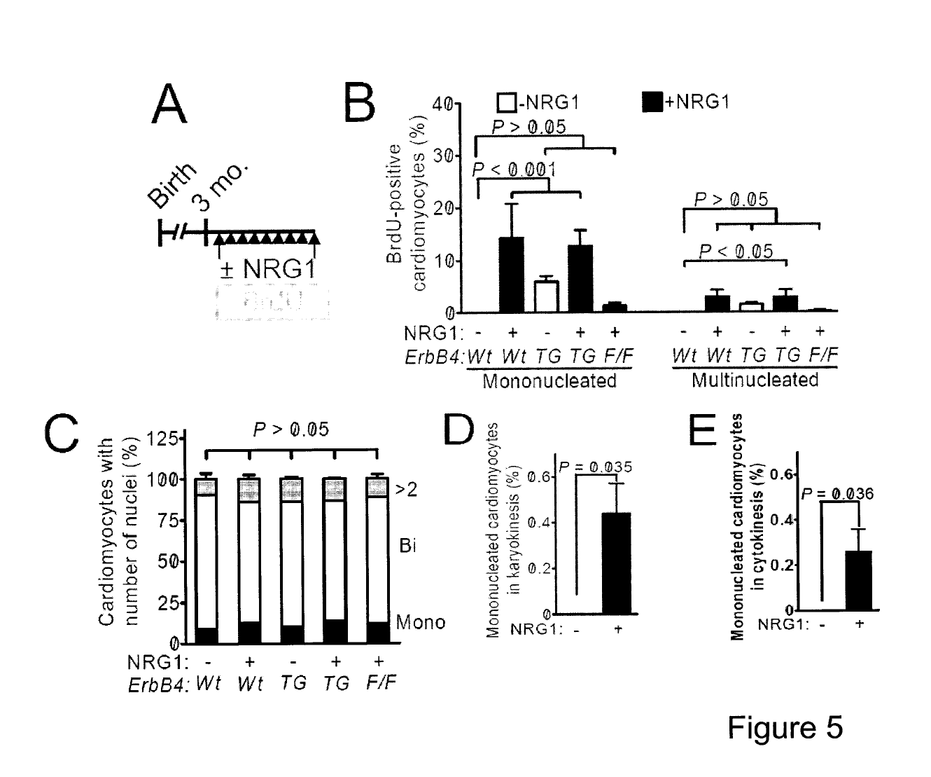

Cardiomyocytes in adult mammalian hearts do not proliferate under baseline conditions. To determine whether stimulating the NRG1/ErbB4 signalling pathway induces cardiomyocyte cell cycle reentry in vivo, we injected recombinant NRG1 in 3 month-old mice, labelled with BrdU in the drinking water for 9 days, and then quantified cardiomyocyte cell cycle activity (FIG. 5A). In control mice, we did not detect cycling cardiomyocytes (FIG. 5B), in accordance with published results. In contrast, in mice injected with recombinant NRG1, 14.3±6.5% of mononucleated and 3±1.2% of multinucleated cardiomyocytes were BrdU-positive (n=3, P<0.001, FIG. 5B). Thus, NRG1 induces differentiated cardiomyocytes to leave proliferative quiescence.

We next determined whether the NRG1-induced cardiomyocyte cell cycle reentry was mediated by ErbB4. Injecting NRG1 into ErbB4-transgenes did not increase cardiomyocyte cycling above the level of injecting NRG1 into littermate controls, suggesting that ErbB4 expression levels were not limiting cardiomyocyte cell cycle reentry (FIG. 5B). We then inactivated ErbB4, which resulted in a marked decrease of NRG1-induced cardiomyocyte DNA synthesis, indicating that ErbB4 was required for NRG1-induced cardiomyocyte cell cycle reentry (FIG. 5B). Importantly, the proportions of mono- and multinucleated cardiomyocytes were not different between the experimental groups (FIG. 5C). We determined whether NRG1 induced cardiomyocyte karyokinesis (FIG. 5D). In NRG1-injected animals, 0.4±0.1% of mononucleated cardiomyocytes were in the process of karyokinesis, but none in controls. We then quantified cardiomyocyte cytokinesis, the terminal phase of the cell cycle (FIG. 5E). In NRG1-injected animals, 0.3±0.1% of mononucleated cardiomyocytes were in the process of cytokinesis, but none in control animals. In summary, activating NRG1/ErbB4 signalling induces quiescent cardiomyocytes to reenter the cell cycle and to undergo karyokinesis and cytokinesis in vivo.

The in vitro optical fate mapping suggested that NRG1 induces differentiated cardiomyocytes to reenter the cell cycle. To test this possibility in vivo, we performed a genetic fate map. Using α-MHC-MerCreMer; Rosa26R mice, we permanently labelled differentiated cardiomyocytes (FIG. 6A). We then injected NRG1 and quantified BrdU uptake. We did not detect BrdU-positive cardiomyocytes in the control group. In NRG1-treated animals, in contrast, we identified BrdU-positive cardiomyocytes that were X-gal positive, indicating that they were differentiated before they reentered the cell cycle (FIG. 6B). We also found X-gal positive cardiomyocytes during karyokinesis (FIG. 6C). In conclusion, NRG1 induces differentiated cardiomyocytes to reenter the cell cycle.

Whether differentiated cardiomyocytes can proliferate is a controversial question in cardiovascular biology. To answer this question in vivo, we analyzed the fate of genetically labelled differentiated cardiomyocytes. We induced sparse genetic labelling of differentiated cardiomyocytes in α-MHC-MerCreMer^{+/+}; Rosa26R^{+/-} mice. One week later, we induced cardiomyocyte proliferation by injecting NRG1 for nine days and quantified the number of multicellular X-gal positive clusters of cardiomyocytes. The majority of X-gal positive cardiomyocytes were single (FIG. 6D). NRG1-injected animals (n=9) had 2.1-fold more clusters of two and 35-fold more clusters consisting of four and more cardiomyocytes than control animals (n=10, FIG. 6E). In conclusion, NRG1 induces proliferation of differentiated cardiomyocytes in vivo.

To determine whether cardiomyocytes can undergo successive cell divisions in vivo, we labelled with chlorodeoxyuridine (CldU) for the first 4 days, followed by a 1-day washout period, and then with iododeoxyuridine (IdU) for the final 4 days of NRG1-injections (FIG. 6F). We readily identified cardiomyocytes that were labelled with one thymidine analog, indicating that they were in the cell cycle once (FIG. 6G). We detected mononucleated cardiomyocytes that took up both thymidine analogs, signifying that they had gone through S-phase, completed cytokinesis, and cycled again at least once more (FIG. 6H). We also detected binucleated cardiomyocytes that took up both thymidine analogs (FIG. 6I). Quantification of the frequency of cell cycle transition in NRG1-stimulated hearts showed that 0.6±0.2% of cardiomyocytes took up CldU during the first 4 days and 0.5±0.1% of cardiomyocytes took up IdU during the final 4 days of NRG1-stimulation (FIG. 6J). Of note, 50±0.08% of mononucleated cardiomyocytes that went through the cell cycle during the first 4 days of NRG1-stimulation underwent another round of replication during the final 4 days (FIG. 6J). Of cardiomyocytes that were double labelled, i.e. that had transitioned two cell cycles, 58±5.9% (n=117) were mononucleated, indicating that they completed cytokinesis, consistent with our video microscopy data. Because the presence of both thymidine analogs in a mononucleated cardiomyocyte allowed us to conclude that they underwent cytokinesis at least once, we can determine the frequency of cytokinesis, which was 0.15±0.04% of all cardiomyocytes over a period of 4 days in NRG1-injected animals, but zero in control animals. In conclusion, NRG1 stimulates a significant portion of mononucleated cardiomyocytes to replicate.

Cytokinesis is a particular challenge for differentiated cardiomyocytes because they contain contractile fibrils organized in sarcomeres. This raises an important question: how do differentiated cardiomyocytes divide their nuclei and cell bodies? To address this question, we visualized the sarcomeric structure in dividing cardiomyocytes. During karyokinesis, the sarcomeric Z-disks and M-bands were disassembled in the region of the midzone (FIG. 6K). Notably, in cytokinesis, the sarcomeric structure was absent from the division plane (FIG. 6L). In conclusion, cardiomyocyte division is associated with sarcomere disassembly.

### Example 5

### Undifferentiated Progenitor Cells do not Contribute to NRG1-Induced Cardiomyocyte Proliferation

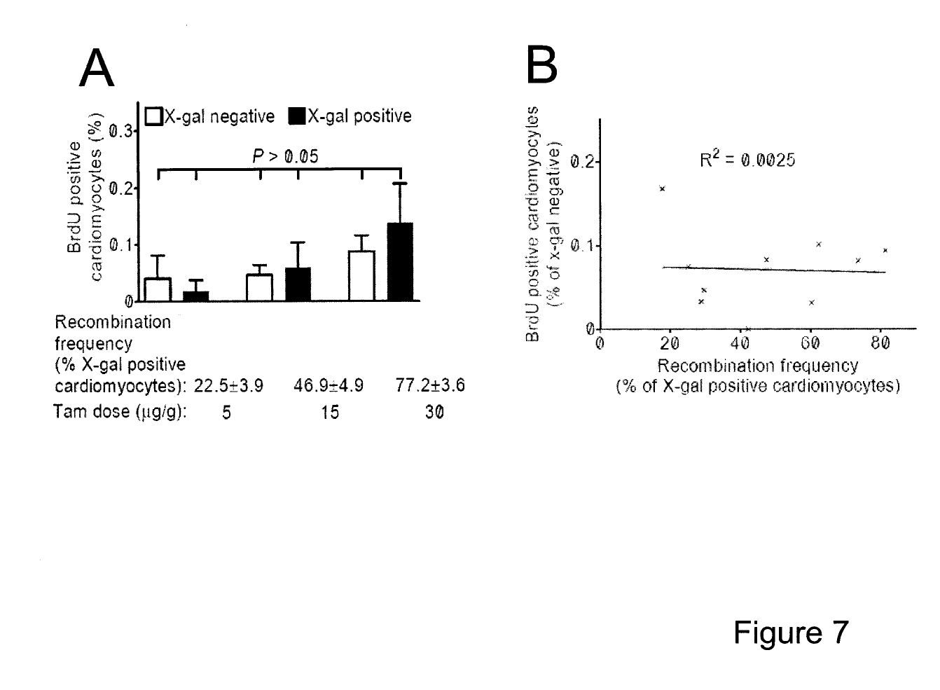

Our results thus far indicated that NRG1 induces differentiated cardiomyocytes to proliferate. However, generation of cardiomyocytes from undifferentiated progenitor cells may also contribute to the observed effect. If NRG1-induced cardiomyocyte proliferation had two different cellular origins, i.e. stemmed from differentiated cardiomyocytes and from undifferentiated progenitor cells, then there should be detectable differences between both processes. We labelled differentiated cardiomyocytes genetically by activating the α-MHC-MerCreMer^{+/+}; Rosa26R^{+/+} system. A hallmark of cardiomyocytes derived from undifferentiated stem or progenitor cells is that they do not carry the α-MHC promoter-dependent genetic label. Thus, genetically labelled cardiomyocytes are derived from differentiated cardiomyocytes. In contrast, cardiomyocytes without the genetic label could be derived from differentiated cardiomyocytes or from undifferentiated progenitor cells. After applying the genetic label, we induced proliferation with NRG1, and labelled with BrdU to visualize newly generated cardiomyocytes. To determine the contribution of progenitor cells to NRG1-induced cardiomyocyte proliferation, we compared BrdU positive, X-gal positive (cardiomyocyte-derived) with BrdU positive, X-gal negative cardiomyocytes (possibly progenitor cell-derived). The morphology of newly generated X-gal positive and X-gal negative cardiomyocytes was identical (FIG. 7A). We visualized cardiomyocytes that were presently in the cell cycle with H3P-staining and compared X-gal positive with X-gal negative cardiomyocytes, which were identical (FIG. 7B). These results suggested that X-gal positive and X-gal negative BrdU positive cardiomyocytes had the same cellular origin, i.e. they originated from differentiated cardiomyocytes.

Because differentiated cardiomyocytes and undifferentiated progenitor cells may have different proliferative rates, we compared the proportion of BrdU-positive cardiomyocytes derived from either origin. Cardiomyocytes stemming from differentiated cardiomyocytes are BrdU positive and X-gal positive and cardiomyocytes stemming from undifferentiated progenitor cells are BrdU positive and X-gal negative. We compared the percentage of BrdU-positive cardiomyocytes across a range of genetic labelling frequencies (FIG. 7C, D). We found that the proliferative rates were identical in X-gal negative and X-gal positive cardiomyocytes at low, intermediate, and high genetic labelling frequencies, indicating that NRG1-induced cardiomyocyte proliferation stemmed from a single cellular source (FIG. 7D).

If there was an influx of cardiomyocytes from undifferentiated progenitor cells, then the measured proliferative rate in this portion should increase with increasing fraction of X-gal positive cardiomyocytes. Thus, we correlated the proliferative rate of X-gal negative cardiomyocytes with the genetic labelling frequency (FIG. 7E). We found no correlation between the proliferative rate of X-gal negative cardiomyocytes and the X-gal labelling frequency, thus excluding the influx of cardiomyocytes from undifferentiated progenitor cells. In summary, NRG1-induced cardiomyocyte proliferation has a single source, which stems from differentiated cardiomyocytes.

### Example 6

### NRG1 Improves Cardiac Function and Structure after Myocardial Infarction

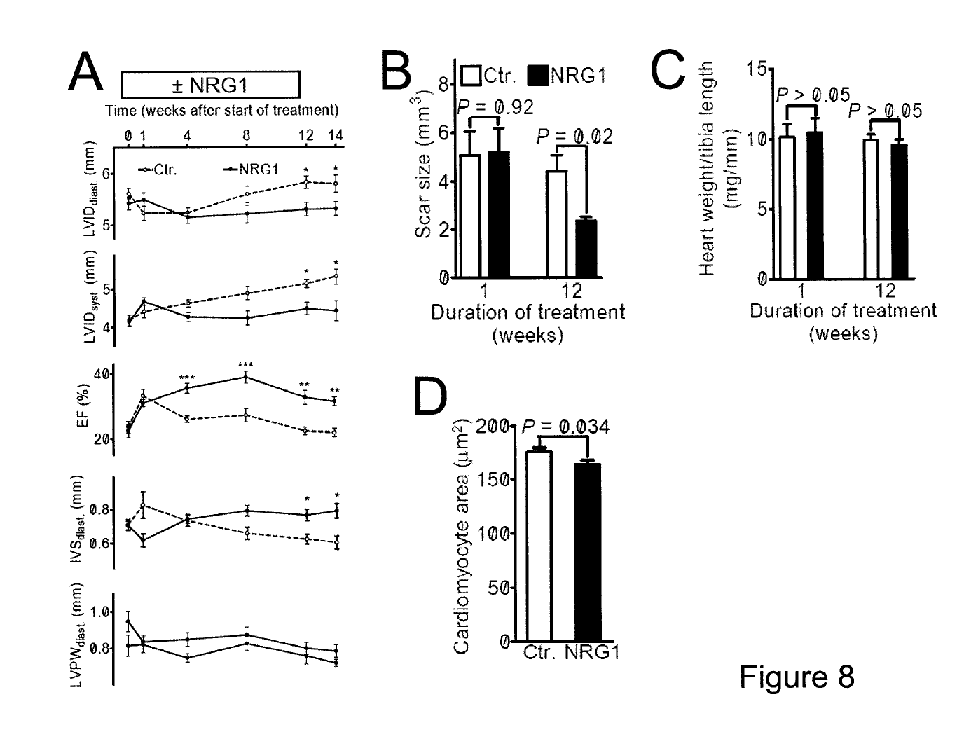

To determine whether inducing cardiomyocyte proliferation with NRG1 is beneficial after myocardial injury, we permanently ligated the left anterior descending coronary artery (LAD) in 2 months-old mice and began daily NRG1-injections one week later for 12 weeks. Because regeneration would be anticipated to produce a permanent structural change, we introduced a two-week window of no injections before the end of the experiment (FIG. 8A). We determined myocardial structure and function by serial echocardiography (FIG. 8B). Over the course of the 15 week-experiment, NRG1-injected animals had no significant increase of the dimension of the left ventricle, whereas control animals had significant left ventricular dilatation, indicating a positive effect of NRG1 on post-infarction remodelling. Injecting NRG1 induced a sustained improvement of myocardial function, determined by ejection fraction. Compensatory hypertrophy, determined by measuring the thickness of the interventricular septum and the left ventricular free wall, was significantly attenuated in NRG1-injected animals. In summary, NRG1 induced sustained improvements after myocardial infarction.

To analyze at the tissue level how NRG1 improved myocardial function, we determined the size of the infarct scar (FIG. 8C). After one week of treatment, control and NRG1-treated hearts had the same scar size, however, after 12 weeks of treatment and two additional weeks without treatment, NRG1-injected animals had a 46%-smaller infarct scar (FIG. 8D). We then compared the heart weight, which was identical at the beginning and two weeks after completion of treatment (FIG. 8E). The cardiomyocyte cross-sectional area, however, was smaller in NRG1-treated hearts, consistent with attenuation of cardiomyocyte hypertrophy (FIG. 8F). Collectively, these results indicate that administration of NRG1 for 12 weeks results in permanently improved myocardial function, smaller infarct scar size, and attenuated myocardial hypertrophy.

### Example 7

### NRG1 Promotes Replacement of Cardiomyocytes after Myocardial Infarction

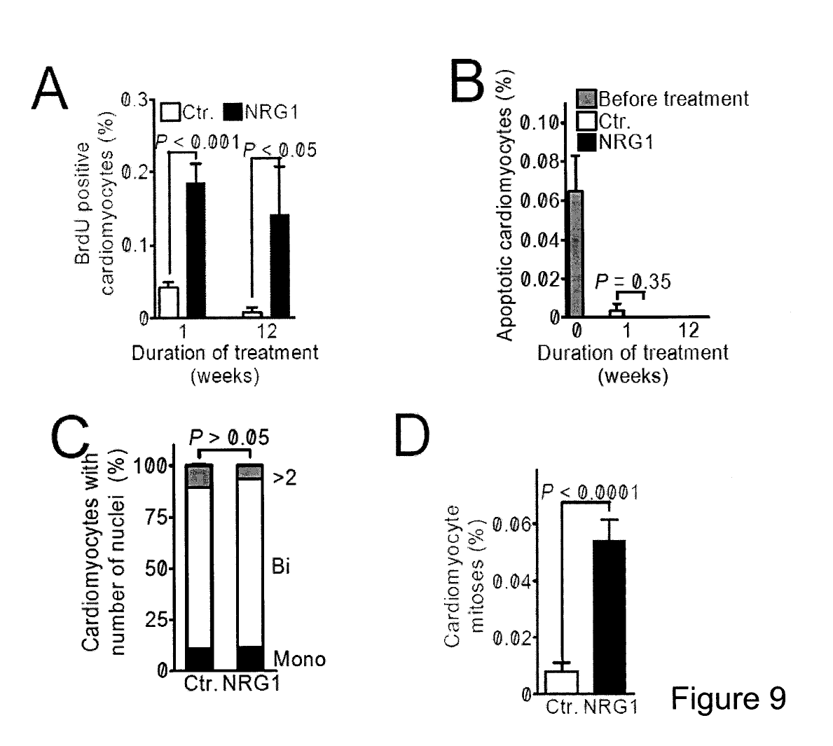

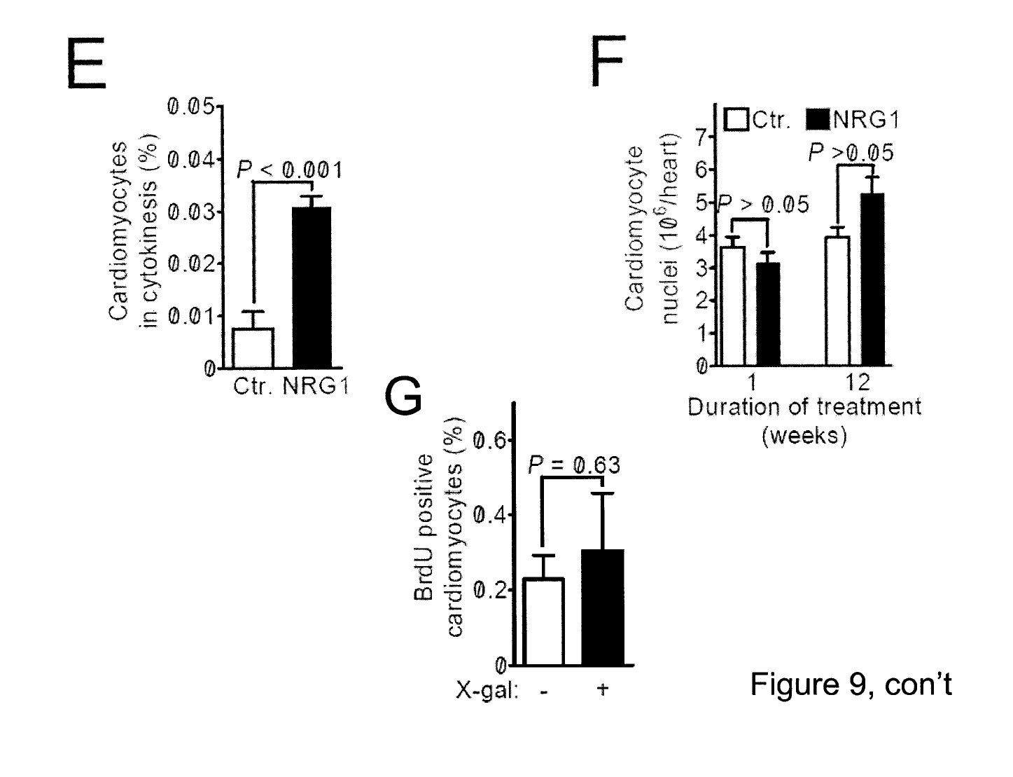

What are the underlying cellular mechanisms for the NRG1-induced improvements? To determine whether the decreased infarct scar size correlated with cardiomyocyte cell cycle reentry, we quantified cardiomyocyte BrdU-uptake. NRG1 increased cardiomyocyte BrdU-uptake 4.4-fold to 0.18±0.03% without affecting cardiomyocyte apoptosis or changing the percentage of mono- and binucleated cardiomyocytes (FIG. 9A-C). Thus, NRG1 increased cardiomyocyte cell cycle activity after myocardial infarction.

Can NRG1-induced cardiomyocyte cell cycle activity account for the observed improvements? By visualizing cardiomyocytes in metaphase, we determined that 2,043 cardiomyocytes per heart were in karyokinesis (FIG. 9D). In conjunction with the duration of karyokinesis of 1.8±0.3 hr, determined by video microscopy (n=18), this would result in 2.3×10⁶ replaced cardiomyocyte nuclei over 12 weeks. We also quantified the portion of cardiomyocytes with a contractile ring (FIG. 9E), which, in conjunction with a duration of cytokinesis of 1.6±0.3 hr (n=10), would result in 722,610 new cardiomyocytes in NRG1-injected animals over 12 weeks. We then quantified cardiomyocyte nuclei directly. NRG1-treated hearts had 1.4×10⁶ more cardiomyocyte nuclei, equivalent to 690,000 more cardiomyocytes after 12 weeks (FIG. 9F). Thus, NRG1-induced cardiomyocyte proliferation can account for the observed cardiomyocyte replacement.

To determine the cellular source of NRG1-induced cardiomyocyte replacement after injury, we permanently labelled differentiated cardiomyocytes with a genetic tag. We found that cycling cardiomyocytes that were genetically labelled were identical in morphology and proliferative rate to unlabelled cardiomyocytes (FIG. 9G,FIG. 10), indicating that undifferentiated stem and progenitor cells did not contribute to NRG1 induced cardiomyocyte replacement.

Bone marrow-derived c-kit positive progenitor cells are required for the endogenous repair process after myocardial infarction. The frequency of c-kit positive cells was identical in control and in NRG1-injected animals, suggesting that NRG1 did not affect recruitment of c-kit positive cells.

While the present invention has been described in terms of specific methods and compositions, it is understood that variations and modifications will occur to those skilled in the art upon consideration of the present invention. Those skilled in the art will appreciate, or be able to ascertain using no more than routine experimentation, further features and advantages of the invention based on the above-described embodiments. The practice of the present invention will employ and incorporate, unless otherwise indicated, conventional techniques of cell biology, cell culture, molecular biology, microbiology, genetic engineering, and immunology, which are within the skill of the art. While the present invention is described in connection with what is presently considered to be the most practical and preferred embodiments, it should be appreciated that the invention is not limited to the disclosed embodiments, and is intended to cover various modifications and equivalent arrangements included within the spirit and scope of the claims. Modifications and variations in the present invention may be made without departing from the novel aspects of the invention as defined in the claims. Accordingly, the invention is not to be limited by what has been particularly shown and described. All publications and references are herein expressly incorporated by reference in their entirety.

What is claimed is:

1. A method of inducing division of a post mitotic heart muscle cell, the method comprising administering a neuregulin composition to the heart muscle cell in an amount effective to stimulate mitotic division of the heart muscle cell.

2. The method of claim 1, wherein the step of administering the neuregulin composition further comprises administering at least a fragment of the neuregulin composition of SEQ ID NO:1 or a sequence that is at least 80% identical to that of the SEQ ID NO:1 fragment.

3. The method of claim 1, wherein the step of administering the neuregulin composition further comprises administering a polypeptide comprising the neuregulin fragment of SEQ ID NO:2 or a functional variant or a sequence that is at least 80% identical to that of the SEQ ID NO:2 fragment.

4. The method of claim 1, wherein the step of administering the neuregulin composition further comprises administering a polypeptide comprising the neuregulin fragment of SEQ ID NO:3 or a functional variant or a sequence that is at least 80% identical to that of the SEQ ID NO:3 fragment.

5. The method of claim 1, wherein the step of administering the neuregulin composition further comprises administering a polypeptide comprising the neuregulin fragment of SEQ ID NO:4 or a functional variant or a sequence that is at least 80% identical to that of the SEQ ID NO:4 fragment.

6. The method of claim 1, wherein the step of administering the neuregulin composition further comprises administering a polypeptide comprising the neuregulin fragment of SEQ ID NO:5 or a functional variant or a sequence that is at least 80% identical to that of the SEQ ID NO:5 fragment.

7. The method of claim 1, wherein the step of administering the neuregulin composition further comprises administering a polypeptide comprising the neuregulin fragment of SEQ ID NO:6 or a functional variant or a sequence that is at least 80% identical to that of the SEQ ID NO:6 fragment.

8. The method of claim 1, wherein the step of administering the neuregulin composition further comprises administering a polypeptide comprising at least an epidermal growth factor-like domain of neuregulin.

9. The method of claim 1, wherein the neuregulin composition activates ErbB4.

10. The method of claim 1, wherein the neuregulin composition induces heterodimerization of ErbB4 and ErbB2 receptors.

11. The method of claim 1, wherein the step of administering the neuregulin composition comprises administering the composition over a duration sufficient to induce cell cycle re-entry of the heart muscle cells.

12. The method of claim 1, wherein the step of administering the neuregulin composition comprises administering the composition over a duration of at least 12 weeks.

13. The method of claim 1, wherein the step of stimulating division further comprises inducing the heart muscle cell to reenter the cell cycle, increase DNA synthesis and induce cytokinesis in the heart muscle cell.

14. A method of inducing proliferation of cardiomyocytes comprising the steps of:
selecting differentiated cells from a tissue that includes said cells;
resuspending said differentiated cells in a growth medium containing an effective amount of a neuregulin composition; and culturing said resuspended cells in the growth medium for a time and under conditions to induce proliferation of at least a portion of said selected cells in culture,
wherein at least a portion of said selected terminally differentiated cells in culture undergo at least one round of cardiomyocyte division.

15. The method of claim 14, wherein the neuregulin composition comprises at least an epidermal growth factor-like domain of neuregulin.

16. The method of claim 14, wherein the method further comprises seeding the cardiomyocytes on a biodegradable scaffold.

17. The method of claim 14, wherein the method further comprises transplanting the proliferating cells into a target area in a subject.

18. The method of claim 17, wherein the target area is a damaged heart tissue.

19. The method of claim 14, wherein the method further comprises incorporating the proliferating cells into a heart tissue implant.

20. The method of claim 19, wherein the method further comprises transplanting the heart tissue implant into a target area in a subject.

21. The method of claim 20, wherein the target area is a damaged heart tissue.

22. A pharmaceutical composition to treat damaged heart tissue comprising an effective amount of a neuregulin composition and a pharmaceutically acceptable carrier, diluent or medium.

23. The composition of claim 22, wherein the neuregulin composition comprises at least an epidermal growth factor-like domain of neuregulin.

24. The composition of claim 22, wherein the neuregulin composition comprises at least a fragment of the neuregulin composition of SEQ ID NO:1 or a sequence that is at least 80% identical to that of the SEQ ID NO:1 fragment.

25. The composition of claim 22, wherein the neuregulin composition comprises the neuregulin fragment of SEQ ID NO:2 or a functional variant or a sequence that is at least 80% identical to that of the SEQ ID NO:2 fragment.

26. The composition of claim 22, wherein the neuregulin composition comprises the neuregulin fragment of SEQ ID NO:3 or a functional variant or a sequence that is at least 80% identical to that of the SEQ ID NO:3 fragment.

27. The composition of claim 22, wherein the neuregulin composition comprises the neuregulin fragment of SEQ ID NO:4 or a functional variant or a sequence that is at least 80% identical to that of the SEQ ID NO:4 fragment.

28. The composition of claim 22, wherein the neuregulin composition comprises the neuregulin fragment of SEQ ID NO:5 or a functional variant or a sequence that is at least 80% identical to that of the SEQ ID NO:5 fragment.

29. The composition of claim 22, wherein the neuregulin composition comprises the neuregulin fragment of SEQ ID NO:6 or a functional variant or a sequence that is at least 80% identical to that of the SEQ ID NO:6 fragment.

30. The composition of claim 22, wherein the composition is formulated for parenteral administration.

31. The composition of claim 22, wherein the composition is formulated for administration with a slow controlled release delivery system.

32. A method of repairing heart tissue, the method comprising
identifying a subject in need of heart tissue repair,
administering to the subject an effective amount of a neuregulin composition, in an amount and regime effective to stimulate division of post-mitotic cardiomyocytes, and
inducing proliferation of the cardiomyocytes to thereby repair heart tissue.

33. The method of claim 32, wherein the step of identifying a subject comprises identifying a subject having experienced at least one of a myocardial ischemia, a hypoxia, a stroke, a myocardial infarction and a chronic ischemic heart disease.

34. The method of claim 32, wherein the step of administering further comprises administering at least an epidermal growth factor-like domain of neuregulin.

35. The method of claim 32, wherein the step of administering further comprises delivering a neuregulin composition by a route selected from the group consisting of a parenterally, an orally, an intraperitoneally, an intravenously, a catheter infusion, an inhalation and a transdermal application.

36. The method of claim 32, wherein the step of delivering further comprises delivering the neuregulin composition locally to the heart tissue.

37. The method of claim 32, wherein the step of delivering further comprises delivering the neuregulin composition with a slow controlled release delivery system.

38. The method of claim 32, wherein the step of administering further comprises administering about 1 mg/kg of a neuregulin composition.

39. The method of claim 32, wherein step of administering further comprises administering the composition over a duration sufficient to induce cardiomyocyte cell cycle re-entry.

40. The method of claim 39, wherein the step of administering neuregulin comprises a duration of at least 12 weeks.

41. The method of claim 39, wherein the step of inducing proliferation further comprises inducing the cardiomyocytes to reenter cell cycle, increasing DNA synthesis and inducing cytokinesis in the cardiomyocytes.

42. The method of claim 39, wherein the step of inducing proliferation further comprises replacing damaged heart tissue with proliferating cardiomyocytes.

43. The method of claim 39, wherein the step of inducing proliferation further comprises improving myocardial function in the subject.

44. The method of claim 39, wherein the step of inducing proliferation further comprises reducing myocardial hypertrophy.

45. A method of treating a condition or disease state by stimulating proliferation of post-mitotic cells comprising administering a compound comprising a neuregulin composition or a pharmaceutically acceptable derivative thereof, whereby the compound treats the condition or disease state by stimulating proliferation of the post-mitotic cells.

46. The method of claim 45, wherein the post-mitotic cells are cardiomyocytes.

47. The method of claim 45, wherein the condition or disease is at least one of a myocardial ischemia, a hypoxia, a stroke, a myocardial infarction and a chronic ischemic heart disease.

48. The method of claim 45, wherein proliferation comprises cell cycle reentry, increased cardiomyocyte DNA synthesis and cytokinesis.
